(19) United States
(12) Patent Application Publication
(10) Pub. No.: US 2022/0249420 A1
(43) Pub. Date: Aug. 11, 2022

(54) MONOUNSATURATED FATTY ACID COMPOSITION AND USE FOR TREATING FATTY LIVER DISEASE

(71) Applicant: Burke & Boyer, Inc., Brooklyn, NY (US)

(72) Inventors: Damion J. Boyer, Cleveland, OH (US); John M. Burke, Concord Township, OH (US)

(73) Assignee: Burke & Boyer, Inc., Brooklyn, NY (US)

(21) Appl. No.: 17/675,507

(22) Filed: Feb. 18, 2022

Related U.S. Application Data
(63) Continuation of application No. PCT/US2020/047491, filed on Aug. 21, 2020.
(60) Provisional application No. 62/889,826, filed on Aug. 21, 2019.

(51) Int. Cl. A61K 31/201; A23L 33/00; A23L 33/12; A61K 47/44; A61P 1/16

(52) U.S. Cl. CPC A61K 31/201; A23L 33/40; A23V 2002/00; A61K 47/44; A61P 1/16; A23L 33/12

(57) ABSTRACT

Methods for treating liver disease and, in particular, fatty liver disease, by administering a composition comprising a mixture of a first monounsaturated fatty acid and a second monounsaturated fatty acid are provided herewith.

### RELATED APPLICATIONS

This application is a continuation application of International Application No. PCT/US2020/047491, filed on Aug. 21, 2020, which in turn claims the benefit of priority to U.S. Provisional Application No. 62/889,826, filed on Aug. 21, 2019. The entire contents of each of the foregoing applications are incorporated herein by reference.

### FIELD OF THE INVENTION

The present invention relates to compositions comprising monounsaturated fatty acids (MUFAs) having low melting points and high iodine values for treating liver diseases, for example, a fatty liver disease, such as non-alcoholic fatty liver disease (NAFLD) and, in particular, non-alcoholic steatohepatitis (NASH).

### BACKGROUND OF THE INVENTION

Metabolic functions of the liver are critical for homeostatic molecule synthesis and energy regulation, and include carbohydrate, fat, and protein metabolism. Fatty liver diseases are conditions in which fat builds up in the liver. There are two main types: nonalcoholic fatty liver disease (NAFLD) and alcoholic fatty liver disease, also called alcoholic steatohepatitis. NAFLD is a type of fatty liver diseases that are not related to heavy alcohol use. NAFLD includes two kinds. One kind is simple fatty liver, which is characterized with fat in the liver but little or no inflammation or liver cell damage. Simple fatty liver typically does not get bad enough to cause liver damage or complications. The other kind is nonalcoholic steatohepatitis (NASH), which is characterized with inflammation and liver cell damage, as well as fat in the liver Inflammation and liver cell damage can cause fibrosis, or scarring, of the liver. NASH may lead to cirrhosis or liver cancer. NASH (sometimes called steatonecrosis) is diagnosed most often in patients between 40 and 60 years of age but can occur in all age groups. Many affected patients have obesity, type 2 diabetes mellitus (or glucose intolerance), dyslipidemia, and/or metabolic syndrome. The only widely accepted treatment is to eliminate potential causes and risk factors. Such treatment may include discontinuation of drugs or toxins, weight loss, and treatment for dyslipidemia or treatment for hyperglycemia. Preliminary evidence suggests that thiazolidinediones and vitamin E can help correct biochemical and histologic abnormalities in NASH. Many other treatments (e.g., ursodeoxycholi acid, metronidazole, metformin, betaine, glucagon, glutamine infusion) have not been proved effective.

There remains a need to develop novel treatment of a fatty liver disease, including NAFLD or NASH, as well as novel treatment and prevention of liver inflammation, liver cell injury, hepatic fibrosis, cirrhosis and liver cancer, e.g., hepatocarcinoma, due to NASH.

### SUMMARY OF THE INVENTION

Disclosed herein are novel compositions comprising MUFAs having low melting point temperatures and, optionally, iodine values of about 50 to about 130; and methods for treating a liver disease in a subject with the compositions.

Accordingly, in a first aspect, the present invention provides a method of treating or preventing a liver disease in a subject comprising administering a composition comprising a mixture of a first monounsaturated fatty acid and a second monounsaturated fatty acid, wherein the mixture has a melting point temperature of about −1.7° C. to about 10.7° C. In one embodiment, the mixture has a melting point temperature of about 3° C. to about 9° C. In another embodiment, the mixture has a melting point temperature of about 7° C. to about 9° C.

In various embodiments of the first aspect of the invention delineated herein, the mixture comprises at least 80% by weight of the composition.

In various embodiments of the first aspect of the invention delineated herein, each of the first monounsaturated fatty acid and the second monounsaturated fatty has an iodine value of about 50 to about 130. In one embodiment, each of the first monounsaturated fatty acid and the second monounsaturated fatty has an iodine value of about 68 to about 130.

In various embodiments of the first aspect of the invention delineated herein, the first monounsaturated fatty acid has an acyl chain of 16 carbons or less.

In various embodiments of the first aspect of the invention delineated herein, the second monounsaturated fatty acid has an acyl chain of 18 carbons or more.

In various embodiments of the first aspect of the invention delineated herein, the composition further comprises one or more additional monounsaturated fatty acids.

In various embodiments of the first aspect of the invention delineated herein, the first monounsaturated fatty acid is selected from the group consisting of C12:1, C14:1 and C16:1. In one embodiment, the first monounsaturated fatty acid is C16:1. In another embodiment, the first monounsaturated fatty is C16:1n-7, C16:1n-6, C16:1n-5, C16:1n-4 or C16:1n-3.

In various embodiments of the first aspect of the invention delineated herein, the second monounsaturated fatty acid is selected from the group consisting of C22:1, C20:1 and C18:1. In one embodiment, the second monounsaturated fatty acid is C18:1. In another embodiment, the second monounsaturated fatty acid is C18:1n-9.

In various embodiments of the first aspect of the invention delineated herein, the double bond in the first monounsaturated fatty acid is a cis-isomer.

In various embodiments of the first aspect of the invention delineated herein, the double bond in the second monounsaturated fatty acid is a cis-isomer.

In various embodiments of the first aspect of the invention delineated herein, the first monounsaturated fatty acid is palmitoleic acid. In further embodiments, the second monounsaturated fatty acid is oleic acid. In a particular embodiment, the first monounsaturated fatty acid is palmitoleic acid and the second monounsaturated fatty acid is oleic acid.

In various embodiments of the first aspect of the invention delineated herein, the first monounsaturated fatty acid is present at up to about 40% by weight of the composition. In one embodiment, the first monounsaturated fatty acid is present at about 18% to about 40% by weight of the composition. In another embodiment, the first monounsaturated fatty acid is present at about 20% to about 40% by weight of the composition. In still another embodiment, the first monounsaturated fatty acid is present at about 18% to about 23% by weight of the composition. In yet another embodiment, the first monounsaturated fatty acid is present at about 20% to about 23% by weight of the composition.

In various embodiments of the first aspect of the invention delineated herein, the second monounsaturated fatty acid is present at up to about 80% by weight of the composition. In one embodiment, the second monounsaturated fatty acid is present at about 40% to about 80% by weight of the composition. In another embodiment, the second monounsaturated fatty acid is present at about 60% to about 80% by weight of the composition.

In various embodiments of the first aspect of the invention delineated herein, the first monounsaturated fatty acid is present at about 18% to about 40% by weight of the composition and the second monounsaturated fatty acid is present at about 40% to about 80% by weight of the composition. In one embodiment, the first monounsaturated fatty acid is present at about 20% to about 40% by weight of the composition and the second monounsaturated fatty acid is present at about 60% to about 80% by weight of the composition. In another embodiment, the first monounsaturated fatty acid is present at about 18% to about 23% by weight of the composition and the second monounsaturated fatty acid is present at about 60% to about 80% by weight of the composition. In still another embodiment, the first monounsaturated fatty acid is present at about 20% to about 23% by weight of the composition and the second monounsaturated fatty acid is present at about 60% to about 80% by weight of the composition.

In various embodiments of the first aspect of the invention delineated herein, the composition further comprises up to about 5%, about 10%, about 15%, about 20%, about 25% or about 30% by weight of saturated fatty acid and polyunsaturated fatty acid; or between about 5% and about 30%, between about 10% and about 25% by weight of saturated fatty acid and polyunsaturated fatty acid or between about 5% and about 20% by weight of saturated fatty acid and polyunsaturated fatty acid.

In various embodiments of the first aspect of the invention delineated herein, the saturated fatty acids is selected from the group consisting of C16:0, C12:0, C14:0 and C10:0. In one embodiment, the saturated fatty acid is up to about 10%, about 15%, about 20% or about 25% by weight; or between about 2% and about 25%, between about 5% and about 20% by weight, or between about 3% and about 10% by weight. In another embodiment, the polyunsaturated fatty acid is selected from the group consisting of C18:2, C18:3, C20:4, C20:5, C22:5 and C22:6. In still another embodiment, the polyunsaturated fatty acid is up to about 10%, about 15% or about 20% by weight; between about 2% and about 20%, between about 3% and about 15% by weight, or between about 3% and about 10% by weight.

In various embodiments of the first aspect of the invention delineated herein, the composition comprises: about 10% to about 40% by weight of C16:1; about 40% to about 80% by weight of C18:1; up to about 15% by weight of saturated fatty acids; and up to about 7% by weight of polyunsaturated fatty acids. In one embodiment, the composition comprises: about 20% by weight of C16:1; about 60% by weight of C18:1; about 15% by weight of C16:0; and about 5% by weight of C18:2.

In various embodiments of the first aspect of the invention delineated herein, the composition comprises: about 10% to about 30% by weight of C16:1; about 60% to about 80% by weight of C18:1; up to about 7% by weight of saturated fatty acids; and up to about 7% by weight of polyunsaturated fatty acids. In one embodiment, the composition comprises: about 20% by weight of C16:1; about 70% by weight of C18:1; about 5% by weight of C16:0; and about 5% by weight of C18:2.

In various embodiments of the first aspect of the invention delineated herein, the composition further comprises up to about 3% by weight trace fatty acids.

In various embodiments of the first aspect of the invention delineated herein, the composition is a liquid at room temperature.

In various embodiments of the first aspect of the invention delineated herein, the liver disease is a fatty liver disease. In one embodiment, the liver disease is NAFLD. In another embodiment, the fatty liver disease is NASH.

In various embodiments of the first aspect of the invention delineated herein, the liver disease is liver inflammation, liver cell injury, hepatic fibrosis, cirrhosis, or liver cancer. In one embodiment, the liver cancer is hepatocarcinoma.

In various embodiments of the first aspect of the invention delineated herein, the composition is administered at a daily dose of about 0.01 to about 2 g, about 0.1 to about 1.5 g, or about 0.3 to about 1.3 g per kilogram of body weight. In one embodiment, the composition is administered at a daily dose of 0.2 to 0.85 g per kilogram of body weight. Alternatively, the composition is administered at a daily dose of about 0.5 grams to about 60 grams, about 5 grams to 55 grams, about 10 grams to 50 grams or about 15 grams to about 45 grams. In one embodiment, the composition is administered at a daily dose of about 2 grams, about 10 grams, about 20 grams, about 30 grams, about 40 grams, about 50 grams or about 60 grams. In another embodiment, the composition is administered at a daily dose of about 1 gram, about 5 grams, about 10 grams, about 15 grams, about 20 grams, about 25 grams, about 30 grams, about 35 grams, about 40 grams, about 45 grams, about 50 grams or about 55 grams.

In various embodiments of the first aspect of the invention delineated herein, the composition is administered for at least 8 weeks.

In various embodiments of the first aspect of the invention delineated herein, the composition is administered with one or more fatty acids or wherein the composition further comprises one or more carrier fatty acids. In one embodiment, the carrier fatty acids are selected from the group consisting of safflower oil, olive oil, palm oil, soybean oil, canola oil (rapeseed oil), corn oil, peanut oil, other vegetable oils and a combination thereof.

In a second aspect, the present invention provides a composition comprising a mixture of a first monounsaturated fatty acid and a second monounsaturated fatty acid, wherein the mixture has a melting point temperature of about −1.7° C. to about 10.7° C. In one embodiment, the mixture has a melting point temperature of about 3° C. to about 9° C. In another embodiment, the mixture has a melting point temperature of about 7° C. to about 9° C.

In various embodiments of the second aspect of the invention delineated herein, the mixture comprises at least 80% by weight of the composition.

In various embodiments of the second aspect of the invention delineated herein, each of the first monounsaturated fatty acid and the second monounsaturated fatty has an iodine value of about 50 to about 130. In one embodiment, each of the first monounsaturated fatty acid and the second monounsaturated fatty has an iodine value of about 68 to about 130.

In various embodiments of the second aspect of the invention delineated herein, the first monounsaturated fatty acid has an acyl chain of 16 carbons or less.

In various embodiments of the second aspect of the invention delineated herein, the second monounsaturated fatty acid has an acyl chain of 18 carbons or more.

In various embodiments of the second aspect of the invention delineated herein, the composition further comprises one or more additional monounsaturated fatty acids.

In various embodiments of the second aspect of the invention delineated herein, the first monounsaturated fatty acid is selected from the group consisting of C12:1, C14:1 and C16:1. In one embodiment, the first monounsaturated fatty acid is C16:1. In another embodiment, the first monounsaturated fatty is C16:1n-7, C16:1n-6, C16:1n-5, C16:1n-4 or C16:1n-3.

In various embodiments of the second aspect of the invention delineated herein, the second monounsaturated fatty acid is selected from the group consisting of C22:1, C20:1 and C18:1. In one embodiment, the second monounsaturated fatty acid is C18:1. In another embodiment, the second monounsaturated fatty acid is C18:1n-9.

In various embodiments of the second aspect of the invention delineated herein, the double bond in the first monounsaturated fatty acid is a cis-isomer.

In various embodiments of the second aspect of the invention delineated herein, the double bond in the second monounsaturated fatty acid is a cis-isomer.

In various embodiments of the second aspect of the invention delineated herein, the first monounsaturated fatty acid is palmitoleic acid. In further embodiments, the second monounsaturated fatty acid is oleic acid. In a particular embodiment, the first monounsaturated fatty acid is palmitoleic acid and the second monounsaturated fatty acid is oleic acid.

In various embodiments of the second aspect of the invention delineated herein, the first monounsaturated fatty acid is present at up to about 40% by weight of the composition. In one embodiment, the first monounsaturated fatty acid is present at about 18% to about 40% by weight of the composition. In another embodiment, the first monounsaturated fatty acid is present at about 20% to about 40% by weight of the composition. In still another embodiment, the first monounsaturated fatty acid is present at about 18% to about 23% by weight of the composition. In yet another embodiment, the first monounsaturated fatty acid is present at about 20% to about 23% by weight of the composition.

In various embodiments of the second aspect of the invention delineated herein, the second monounsaturated fatty acid is present at up to about 80% by weight of the composition. In one embodiment, the second monounsaturated fatty acid is present at about 40% to about 80% by weight of the composition. In another embodiment, the second monounsaturated fatty acid is present at about 60% to about 80% by weight of the composition.

In various embodiments of the second aspect of the invention delineated herein, the first monounsaturated fatty acid is present at about 18% to about 40% by weight of the composition and the second monounsaturated fatty acid is present at about 40% to about 80% by weight of the composition. In one embodiment, the first monounsaturated fatty acid is present at about 20% to about 40% by weight of the composition and the second monounsaturated fatty acid is present at about 60% to about 80% by weight of the composition. In another embodiment, the first monounsaturated fatty acid is present at about 18% to about 23% by weight of the composition and the second monounsaturated fatty acid is present at about 60% to about 80% by weight of the composition. In still another embodiment, the first monounsaturated fatty acid is present at about 20% to about 23% by weight of the composition and the second monounsaturated fatty acid is present at about 60% to about 80% by weight of the composition.

In various embodiments of the second aspect of the invention delineated herein, the composition further comprises up to about 5%, about 10%, about 15%, about 20%, about 25% or about 30% by weight of saturated fatty acid and polyunsaturated fatty acid; between about 5% and about 30%, between about 10% and about 25% by weight of saturated fatty acid and polyunsaturated fatty acid or between about 5% and about 20% by weight of saturated fatty acid and polyunsaturated fatty acid.

In various embodiments of the second aspect of the invention delineated herein, the saturated fatty acids is selected from the group consisting of C16:0, C12:0, C14:0 and C10:0. In one embodiment, the saturated fatty acid is up to about 10%, about 15%, about 20% or about 25% by weight; or between about 2% and about 25%, between about 5% and about 20% by weight, or between about 3% and about 10% by weight. In another embodiment, the polyunsaturated fatty acid is selected from the group consisting of C18:2, C18:3, C20:4, C20:5, C22:5 and C22:6. In still another embodiment, the polyunsaturated fatty acid is up to about 10%, about 15% or about 20% by weight; or between about 2% and about 20%, between about 3% and about 15% by weight, or between about 3% and about 10% by weight.

In various embodiments of the second aspect of the invention delineated herein, the composition comprises: about 10% to about 40% by weight of C16:1; about 40% to about 80% by weight of C18:1; up to about 15% by weight of saturated fatty acids; and up to about 7% by weight of polyunsaturated fatty acids. In one embodiment, the composition comprises: about 20% by weight of C16:1; about 60% by weight of C18:1; about 15% by weight of C16:0; and about 5% by weight of C18:2.

In various embodiments of the second aspect of the invention delineated herein, the composition comprises: about 10% to about 30% by weight of C16:1; about 60% to about 80% by weight of C18:1; up to about 7% by weight of saturated fatty acids; and up to about 7% by weight of polyunsaturated fatty acids. In one embodiment, the composition comprises: about 20% by weight of C16:1; about 70% by weight of C18:1; about 5% by weight of C16:0; and about 5% by weight of C18:2.

In various embodiments of the second aspect of the invention delineated herein, the composition further comprises up to about 3% by weight trace fatty acids.

In various embodiments of the second aspect of the invention delineated herein, the composition is a liquid at room temperature.

In various embodiments of the second aspect of the invention, the composition further comprises one or more carrier fatty acids, for example, selected from the group consisting of safflower oil, olive oil, palm oil, soybean oil, canola oil (rapeseed oil), corn oil, peanut oil, other vegetable oils and a combination thereof.

In a particular embodiment, the composition is for the treatment or prevention of liver disease and includes a) about 20% by weight of C16:1n-7; b) about 70% by weight of C18:1n-9; c) about 5% by weight of C16:0; and d) about 5% by weight of C18:2. In a particular embodiment, the composition further includes one or more carrier fatty acids selected from the group consisting of safflower oil, olive oil, palm oil, soybean oil, canola oil (rapeseed oil), corn oil, peanut oil, other vegetable oils and a combination thereof. In a specific embodiment, the carrier fatty acid is safflower oil.

Other features and advantages of the invention will be apparent from the following detailed description and drawings.

### BRIEF DESCRIPTION OF THE DRAWINGS

FIG. 1 shows body weight changes in control animals and animals treated with 50% composition A and 50% safflower oil, 25% composition A and 75% safflower oil, or composition A.

FIG. 2A shows food intake (Day 1-Day 2) in control animals and animals treated with composition A. FIG. 2B shows food intake (Day 3-Day 14) in control animals and animals treated with 50% composition A and 50% safflower oil or treated with 25% composition A and 75% safflower oil.

FIG. 3A shows body weight in control animals and animals treated with 50% composition A and 50% safflower oil or treated with 25% composition A and 75% safflower oil. FIG. 3B shows liver weight in control animals and animals treated with 50% composition A and 50% safflower oil or treated with 25% composition A and 75% safflower oil. FIG. 3C shows liver-to-body weight ratio in control animals and animals treated with 50% composition A and 50% safflower oil or treated with 25% composition A and 75% safflower oil.

FIG. 4A shows whole blood glucose in control animals and animals treated with 50% composition A and 50% safflower oil or treated with 25% composition A and 75% safflower oil. FIG. 4B shows plasma ALT in control animals and animals treated with 50% composition A and 50% safflower oil or treated with 25% composition A and 75% safflower oil. FIG. 4C shows liver hydroxyproline in control animals and animals treated with 50% composition A and 50% safflower oil or treated with 25% composition A and 75% safflower oil. FIG. 4D shows liver triglyceride in control animals and animals treated with 50% composition A and 50% safflower oil or treated with 25% composition A and 75% safflower oil. FIG. 4E shows palmitoleic acid (C16:1) in control animals (No. 101) and animals treated with 50% composition A and 50% safflower oil (No. 106) or treated with 25% composition A and 75% safflower oil (No. 108).

FIG. 5A shows representative photomicrographs of HE-stained liver sections from control animals (No. 101) and animals treated with 50% composition A and 50% safflower oil (No. 106) or treated with 25% composition A and 75% safflower oil (No. 108). FIG. 5B shows NAFLD activity score in control animals and animals treated with 50% composition A and 50% safflower oil or treated with 25% composition A and 75% safflower oil. FIG. 5C shows steatosis score in control animals and animals treated with 50% composition A and 50% safflower oil or treated with 25% composition A and 75% safflower oil. FIG. 5D shows inflammation score in control animals and animals treated with 50% composition A and 50% safflower oil or treated with 25% composition A and 75% safflower oil. FIG. 5E shows ballooning score in control animals and animals treated with 50% composition A and 50% safflower oil or treated with 25% composition A and 75% safflower oil. FIG. 5F shows representative photomicrographs of Sirius red-stained liver sections in control animals and animals treated with 50% composition A and 50% safflower oil or treated with 25% composition A and 75% safflower oil. FIG. 5G shows fibrosis area in control animals (No. 101) and animals treated with 50% composition A and 50% safflower oil (No. 106) or treated with 25% composition A and 75% safflower oil (No. 108).

FIG. 6 shows a representative schematic of the study outline to determine the effects of compositions described herein in CDAA-HFD Model of progressive human fibrotic NASH.

FIG. 7A shows a representative schematic of the progression of induced liver disease in DIAMOND mice. FIG. 7B shows a representative schematic of the study outline to determine the effects of compositions described herein in a DIAMOND Model of liver disease.

### DETAILED DESCRIPTION OF THE INVENTION

Disclosed herein are methods of treating a fatty liver disease. The method can include administering a composition comprising a mixture of a first monounsaturated fatty acid and a second monounsaturated fatty acid. The mixture has a melting point temperature of about −1.7° C. to about 10.7° C. In particular embodiments, each of the first monounsaturated fatty acid and the second monounsaturated fatty has an iodine value of 50 to 130.

Saturated fatty acids, such as palmitic (C16:0) and stearic (C18:0), are solids at both room (about 23° C.) and physiological (about 37° C.) temperatures. In contrast, fatty acids such as palmitoleic acid (C16:1), oleic acid (C18:1) and linoleic acid (C18:2) generally having the same length acyl chains as palmitic and stearic acids, but with one or more unsaturated bonds have low melting point temperatures and therefore are liquids at such temperatures. TABLE 1 provides the melting point of some common fatty acids.

TABLE 1

| Fatty Acid | | Melting |
|---|---|---|
| Structure | Fatty Acid Name | Point (° C.) |
| C20:5 | Eicosapentaenoic | −54.0 |
| C22:6 | Docosahexaenoic | −50.0 |
| C4:0 | Butyric | −8.0 |
| C18:2 | Linoleic | −5.0 |
| C18:3 | Linolenic | −5.0 |
| C14:1 | Myristoleic | −4.5 |
| C6:0 | Caproic | −3.4 |
| C16:1 | Palmitoleic | −0.5 |
| C18:1 | Oleic | 13.4 |
| C8:0 | Caprylic | 16.7 |
| C20:1 | Eicosenoic | 23.5 |
| C22:1 | Docosenoic | 30.0 |
| C10:0 | Capric | 31.6 |
| C24:1 | Tetracesenoic | 42.5 |
| C12:0 | Lauric | 44.2 |
| C14:0 | Myristic | 53.9 |
| C16:0 | Palmitic | 63.1 |
| C18:0 | Steric | 69.8 |
| C20:0 | Arachidic | 75.3 |
| C22:0 | Behenic | 79.9 |
| C24:0 | Lignoceric | 84.2 |

Many of the fatty acids found in TABLE 1 can be found as triglycerides in natural sources such as plants, nuts and animals. These natural triglyceride sources, however, contain mixtures of many fatty acids differing in both acyl chain length and degree of saturation. For instance, lard (from pigs), tallow (from cattle) and mutton tallow (from sheep) are solid fats at room temperature, and between 40% and 50% of their acyl groups are saturated C16:0 and C18:0. The monounsaturated oleic acid (C18:1) constitutes about 40% to 50% of their acyl content and much of the remainder is polyunsaturated linoleic acid (C18:2). By contrast, most vegetable oils, which are liquids at room temperature, have only 10% to 20% of palmitic and stearic acid esters, with the remainder being mostly unsaturated oleic and linoleic acid esters.

Iodine value is a measure of the degree of carbon-carbon double bonds of an oil or fat. Saturated (having no carbon-carbon double bonds) oils and fats take up no iodine and therefore their iodine value is zero. In contrast, unsaturated oils and fats take up iodine. The more double bonds present, the more iodine is attached, the higher the iodine value, and the more reactive and unstable the oil or fat becomes. It has been discovered that fatty acids with iodine value above 130, can cross-link and polymerize to form deposits within the body while those fatty acids having an iodine value of about 50 to about 130 are more stable and less prone to polymerization and attachment to the arterial walls.

TABLE 2 provides iodine values for some common fatty acids. In various embodiments, those fatty acids having an iodine value of about 50 to about 130 can be incorporated into the compositions of the present invention.

TABLE 2

| Fatty Acid Structure | Fatty Acid Name | Iodine Value |
|---|---|---|
| C4:0 | Butyric | 0 |
| C6:0 | Caproic | 0 |
| C8:0 | Caprylic | 0 |
| C10:0 | Capric | 0 |
| C12:0 | Lauric | 0 |
| C14:0 | Myristic | 0 |
| C16:0 | Palmitic | 0 |
| C18:0 | Steric | 0 |
| C20:0 | Arachidic | 0 |
| C22:0 | Behenic | 0 |
| C24:0 | Lignoceric | 0 |
| C24:1 | Tetracesenoic | 69 |
| C22:1 | Docosenoic | 74 |
| C20:1 | Eicosenoic | 81 |
| C18:1 | Oleic | 89 |
| C16:1 | Palmitoleic | 99 |
| C14:1 | Myristoleic | 112 |
| C12:1 | Lauroleic | 128 |
| C18:2 | Linoleic | 181 |
| C18:3 | Linolenic | 273 |

The MUFA compositions described herein comprises a first fatty acid group of short carbon chains, optionally, with at least one member selected from the group of C12:1, C14:1, and C16:1 and a second fatty acid group of long carbon chains, optionally, with at least one member selected from the group of C22:1, C20:1, and C18:1.

Preferred MUFA compositions are provided that comprise about 18% to about 40% by weight of the first short carbon chain group and about 40% to about 80% by weight of the second long carbon chain group. Representative examples of the compositions contemplated herein with the ratio concentrations of short carbon chain to the long carbon chain MUFAs are provided in TABLE 3.

TABLE 3

| Short Carbon Chain | Long Carbon Chain |
|---|---|
| MUFA | MUFA |
| 18%-40% | 60%-80% |
| 20%-40% | 60%-80% |
| 18%-23% | 60%-80% |
| 20%-23% | 60%-80% |
| 20%-40% | 40%-80% |

Methods for treating liver diseases, for example, fatty liver diseases such as NAFLD and/or NASH, are also provided by administering the compositions set forth herein. Examples of such methods include administering the compositions described in TABLE 3 to a patient at about 0.01 to about 2 g, about 0.1 to about 1.5 g, or about 0.3 to about 1.3 g per kilogram of body weight, for example, 0.375 g per kilogram of body weight, per day for at least 8 weeks, or 0.125 g per kilogram of body weight, three times a day, per day for at least 8 weeks.

MUFAs have a single vinylic or carbon-carbon double bound along the acyl hydrocarbon chain. Hereinafter, the structure of the fatty acids will be characterized by notations such as Cx:yn-a. The “Cx” indicates that the fatty acyl group contains “x” carbon atoms. The “y” designates the number of carbon-carbon double bonds in the acyl chain and “n-a” designates that the most distal double bond terminates on the “a” the carbon counting from the terminal methyl end.

The MUFAs selected for the present invention include blends or mixtures of fatty acids having low melting point temperature of about −1.7° C. to about 10.7° C. and, optionally, high iodine values of about 50 to about 130. In particular embodiments, methods are provided involving administration of a composition combining high concentrations of at least two groups of the selected MUFA wherein at least one member of the first MUFA group is selected from the short carbon chain MUFA (acyl carbon length of 16 carbons or less) and at least one member of the second group of MUFA is selected from the long carbon chain MUFA (acyl carbon length of 18 carbons or greater). Non-limiting examples of MUFAs contemplated herein can be found in TABLE 4 below. Representative short carbon chain MUFAs include C12:1, C14:1 and C16:1 and long carbon chain MUFAs include C22:1, C20:1 and C18:1. The MUFAs discussed in the present invention may exist in both the cis or trans configuration and both geometric isomers are contemplated in the present invention.

TABLE 4
Monounsaturated Fatty Acid

| Structure | Name |
|---|---|
| C12:1 | Lauroleic |
| C14:1 | Myristoleic |
| C16:1 | Palmitoleic |
| C18:1 | Oleic |
| C20:1 | Eicosenoic |
| C22:1 | Docosenoic |

Of note, although the short chain MUFA palmitoleic acid has been known for many years, it has not been suggested as a useful compound for dietary modification. The advocates of oleic acid as a dietary replacement for PUFAs and saturated fats have not provided similar teachings for the utility of the shorter chain homologues such as palmitoleic acid. Little or no significance has been attributed by the medical or biochemical community to the presence of palmitoleic and myristoleic acids as important constituents of animal lipids.

According to some embodiments, the short carbon chain MUFAs and the long carbon chain MUFAs described herein should comprise at least about 80% by weight of the total composition. Preferably the short carbon chain MUFA group comprises about 18% to about 40% by weight of the composition, more preferably about 18% to about 23% and even more preferably about 20% to about 23% of the composition. The long carbon chain MUFA group preferably comprises about 40% to about 80% and more preferably about 60% to about 80% by weight of the composition.

Preferred fatty acids are C16:1 and C18:1. The fatty acid C16:1 can comprise any one of its positional isomers including C16: 1n-7, C16: 1n-6, C16: 1n-5, C16: 1n-4, and C16: 1n-3 as well as its cis and trans-geometric isomers, or combinations thereof. A more preferred fatty acid is C16: 1n7. The trivial name for C16: 1n7 is palmitoleic acid which is represented generically by Formula I.

CH₃(CH₂)₅CH═CH(CH₂)₇COOH (Formula I)

C16:1 is generally available in naturally occurring oils (“source oils”) although in small amounts. Desired oil fractions containing C16:1 can be readily obtained from various types of oil (i.e., various source oils) such as vegetable oils, seed or nut oils, fish oils, animal fats, or aquatic plants oil, such as salt water or fresh water plants, and certain microbes by conventional cooling and distillation techniques, and/or solvent extraction (“refining” techniques) well known to those skilled in the art. Generally vegetable oils as well as nut or seed oils are not high in the desired content of C16:1. Oil sources that have high amounts of the C16:1 of the present invention include fish oils such as sardine and menhaden that can contain from about 10% to about 16% by weight of C16: 1n-7 whereas sperm whale oil can contain about 13% or more by weight. Although nut oils generally do not have C16:1n-7 fatty acids, an exception is macadamia nut oil that contains C16:1n-7 in amounts of from about 16% to about 25% by total weight of the oil, although it also contains other undesirable fatty acids. Animal fats such as butter oil, chicken fat, lard, and beef tallow generally have high levels of C16: 1n-7. Similarly, they also contain high levels of other undesirable fatty acids. Accordingly, the desired C16: 1n-7 fatty acid can be extracted and isolated from these sources.

For example, the C16: 1n-7 fatty acid as well as the corresponding alcohols, diglycerides, triglycerides and salts can be extracted from the above-noted types of oil (i.e., the source oils) by initially cooling the oil to a temperature below the solidification or melting point temperature of the desired C16:1n-7 and then removing the remaining liquid portion. The removed liquid oil can then be subjected to distillation wherein compounds having higher boiling points than the C16: 1n-7, etc., C14: 1n-5, etc., C12: 1n-3, can be removed. As should be apparent to one skilled in the art, the cooling-distillation process can be repeated until the C16:1 or its corresponding alcohol, diglyceride, triglyceride and/or salt is obtained in concentrated amounts. Alternatively, various one or more solvents can be utilized that dissolve C16:1 (and/or corresponding alcohols, diglycerides, triglycerides and salts) but no other components of the oil so that upon vaporization of the solvent, the selective fatty acid (and/or corresponding alcohols, diglycerides, triglycerides and salts) is obtained. Such techniques and processes are well known in the art. For example, see the description as set forth in U.S. Pat. No. 5,198,250, such as in Example 1 thereof, which is hereby fully incorporated by reference.

By refining the source oil, high or concentrated amounts of C16:1 can be obtained such as from about 10% to about 35% or about 50% or about 75% or about 90% of total fatty acids extracted from the source. Such refined source oils may also contain low amounts of various saturated fatty acid components such as C12:0, C14:0, C16:0, and C18:0, e.g., ≤20 vol. %, ≤15 vol. %, ≤12 vol. %, ≤10 vol. %, and ≤5 vol. %, based on the total amount of fatty acid components in the composition. Similarly, the refined source oils also contain low amounts of polyunsaturated fatty acid oil components such as C12:2 or C12:3, C14:2 or C14:3, C16:2 or C16:3, C18:2 or C18:3, and the like, e.g., <20 vol. %, <15 vol. %, <12 vol. %, <10 vol. %, and even <5 vol. %, based on the total amount of fatty acid components in the composition.

Various strains of algae are suitable with regard to producing the desired source oil for C16:1. The algae can be grown in tanks containing nutrients therein such as phosphates. Numerous strains of algae have relatively high contents of palmitoleic acid such as cyanobacteria, Phormidium sp. NKBG 041105 and Oscillatoria sp. NKBG 091600, that have high cis-palmitoleic acid content (54.5% and 54.4% of total fatty acid, respectively). Phormidium sp. NKBG 041105 has the highest cis-palmitoleic acid content per biomass (46.3 mg (g dry cell weight)−1), and the cis-palmitoleic acid composition was found to be constant with varying temperature. In a similar manner, other aquatic plants such as sea buckthorn can also be grown and utilized. The algae, sea buckthorn, etc., can then be processed by known techniques such as cooling-distillation or solvent extraction to obtain moderate to high concentrations of C16:1n-7 oil fractions. In addition, WO 2009/105620, published Aug. 29, 2009, and WO 2008/036654, published Mar. 27, 2008, identify additional algae suitable as source oil for C16:1. Both publications are hereby fully incorporated by reference.

The fatty acid C18:1 can comprise any of its positional isomers including C18:1n9. Fatty acid C18:1n9, also known as oleic acid, is a preferred fatty acid of C18:1 and is represented generically by Formula II:

CH₃(CH₂)₇CH═CH(CH₂)₇COOH (Formula II)

C18:1 can be extracted from natural sources such as safflower oil or olive oil as described in U.S. Pat. No. 6,664,405 or purchased from commercial sources such as Sea Land Chemical, Cargill, and Emory.

The MUFAs contemplated herein may be used in any form including as a free fatty acid, fatty acid ethyl ester, fatty acid amide or derivatives thereof, salt, monoglyceride, diglyceride, or triglyceride. Any modifications to the MUFAs should result in a physiologically acceptable composition. As used herein, the term “physiologically acceptable” ester or salt means an ester or salt form of the active ingredient that is compatible with the other ingredients of a dietary supplement, nutraceutical or pharmaceutical composition and which is not deleterious to the subject receiving the composition when administered.

As used herein, the term “monoglyceride” refers to a fatty acid covalently bonded to a glycerol molecule through an ester linkage. The term “diglyceride” refers to two fatty acid chains, covalently bonded to a glycerol molecule through an ester linkages. The term “triglyceride” refers to three fatty acid chains covalently bonded to a glycerol molecule through ester linkages. Each of the fatty acid chains bound to the glycerol molecule of the di or triglyceride, may or may not be identical.

The monounsaturated fatty alcohols can be derived from the above MUFAs by reduction thereof by a strong base such as lithium aluminum hydride and secondary separation steps such as, but not limited to, fractional distillation, as known in the art. The fatty alcohol derivative will thus have the same number of total carbon atoms therein, will be monounsaturated, and will contain the double bond at the same location as set forth with regard to the MUFAs discussed herein. Suitable salts of the above MUFAs, or the monounsaturated fatty alcohols, or the monounsaturated fatty acid, mono, di- or triglyceride include the various halides such as chlorine.

The terms “treat” or “treating,” as used herein, refer to partially or completely alleviating, inhibiting, delaying onset of, reducing the incidence of, ameliorating and/or relieving an undesired physiological change or disorder. The terms “preventing” or “prevention” or “prevent” refer to a reduction in risk of acquiring a disease or disorder (i.e., causing at least one of the clinical symptoms of the disease not to develop in a patient that may be exposed to or predisposed to the disease but does not yet experience or display symptoms of the disease).

As used herein, the term “subject” includes a human subject, although it is to be understood that the methods described herein are effective with respect to all vertebrate species, which are intended to be included in the term “subject”. Accordingly, a “subject” can include a human subject for medical purposes, such as for the treatment of an existing condition or disease or the prevention of the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Liver diseases include fatty liver diseases and liver diseases associated therewith. In one embodiment, the liver diseases include NAFLD or NASH.

Pathophysiology of NASH involves fat accumulation (steatosis), inflammation, and, variably, fibrosis. Steatosis results from hepatic triglyceride accumulation. Possible mechanisms for steatosis include reduced synthesis of very low density lipoprotein (VLDL) and increased hepatic triglyceride synthesis (possibly due to decreased oxidation of fatty acids or increased free fatty acids being delivered to the liver). Inflammation may result from lipid peroxidative damage to cell membranes. These changes can stimulate hepatic stellate cells, resulting in fibrosis. If advanced, NASH can cause cirrhosis and portal hypertension.

Therefore, NASH is characterized with inflammation and liver cell damage, as well as fat in the liver. Inflammation and liver cell damage can cause fibrosis, or scarring, of the liver. NASH may lead to cirrhosis or liver cancer. In one embodiment, the liver diseases that would benefit from the treatment with MUFAs include liver inflammation, liver cell injury, hepatic fibrosis, cirrhosis and liver cancer, e.g., hepatocarcinoma, due to NASH.

Most NASH patients are asymptomatic. However, some have fatigue, malaise, or right upper quadrant abdominal discomfort. Hepatomegaly develops in about 75% of patients. Splenomegaly may develop if advanced hepatic fibrosis is present and is usually the first indication that portal hypertension has developed. Patients with cirrhosis due to NASH can be asymptomatic and may lack the usual signs of chronic liver disease. In one embodiment, the liver diseases that would benefit from the treatment with MUFAs include hepatomegaly due to NASH.

The term “composition” as used herein includes therapeutic and dietary formulations. The compositions of the present invention are formulated in the sense that the fatty acid content of the dietary supplement, nutraceutical or pharmaceutical composition is manipulated or adjusted to provide the desired high concentrations of MUFAs having low melting points and, optionally, iodine values of about 50 to about 130, represented by the short and long carbon chain MUFA: C22:1, C20:1, C18:1, C16:1, C14:1 and C12:1. The term “dietary supplement” is a product intended for ingestion that contains a nutrient. The nutrient may be one, or any combination, of the following substances: vitamins, minerals, fiber, fatty acids, or amino acids, among other substances. The term “nutraceutical” is any foodstuff that has physiological activity beyond that of the foodstuff.

The MUFA provided herein is formulated, meaning that the individual components are mixed together from purified sources, to form the high concentrations of short and long carbon chain MUFAs described herein. Alternatively, naturally occurring foodstuffs can be modified to achieve the high composition of MUFAs described herein, exhibiting unexpected and improved therapeutic properties. Specifically, long and short carbon chain MUFAs provided herein are added or supplemented to the foodstuffs to achieve a mixture of MUFA exhibiting the desired melting point, i.e., between −1.7° C. to about 10.7° C.; and/or such that (i) the final short and long carbon chain MUFA concentration in the foodstuff is greater than about 80% by weight total and (ii) the percentage weight of each group of short and long carbon chain MUFA falls within the concentration ranges provided in TABLE 3.

The composition contemplated herein may also be administered to a mammal in the form of a pharmaceutical composition. Such a pharmaceutical composition may contain only the MUFAs contemplated herein as the active ingredients. The pharmaceutical composition may also contain said active ingredients together with one or more pharmaceutically acceptable carriers and any additional ingredients.

A “pharmaceutically acceptable carrier” is meant to encompass any carrier, which does not interfere with effectiveness of the activity of the active ingredient and is not toxic to the host to which it is administered. Pharmaceutically acceptable carriers are known to those of ordinary skill in the art. In one embodiment, pharmaceutically acceptable carriers are carrier fatty acids. The carrier fatty acids are selected from the group consisting of safflower oil, olive oil, palm oil, soybean oil, canola oil (rapeseed oil), corn oil, peanut oil, other vegetable oils and a combination thereof.

“Additional ingredients” means one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Examples of acceptable fillers, sometimes referred to as diluents, include water, sugars such as lactose, dextrose, sucrose, maltose, or microcrystalline cellulose, clays, and mixtures thereof.

Binders that are useful in the present invention include pharmaceutically acceptable substances with cohesive properties. Some examples include celluloses such as hydroxypropyl methycellulose, hydroxypropyl cellulose and carboxymethycellulose sodium, polyvinylpyrrolidone, sugars, starches, and mixtures thereof.

Examples of stabilizing agents that are useful in the present invention include organic acids and alkaline metal salts of organic acids, such as succinic acid, fumaric acid, citric acid, sodium citrate, and mixtures thereof.

Examples of lubricants, glidants and/or antiadherents that may be used in the present invention include talc, magnesium stearate, calcium stearate, stearic acid, hydrogenated vegetable oils, polyethylene glycols, silicon dioxide, and mixtures thereof.

Examples of disintegrating agents that can be used in the present invention include corn starch, croscarmelose sodium, crospovidone (polyplasdone XL-10), sodium starch glycolate (EXPLOTAB® or PRIMOJEL®), or any combination of the foregoing.

Examples of solvents that may be employed are water, methanol, ethanol, isopropyl alcohol, acetone, hexane, butyl alcohol, ethyl acetate, polyethylene glycol, propylene glycol, ethylene glycol, triethylene glycol, glycerin, 1,3-propane diol, 2-methyl-1,3-propane diol, glycerol ricinoleate, mineral oil, peanut oil, corn oil, cottonseed oil, sesame oil or a combination thereof.

Flavoring agents that may be used in the present invention include peppermint, spearmint, wintergreen, cinnamon, coconut, coffee, chocolate, vanilla, menthol, licorice, anise, apricot, caramel, pineapple, strawberry, raspberry, grape, cherry, mixed berry, tropical fruits, mint, and mixtures thereof.

Coloring agents that may be employed in the present invention include FD&C-type dyes and lakes, fruit and vegetable extracts, titanium dioxide, and mixtures thereof

Pharmaceutical compositions described herein may be prepared by any known method or method developed hereafter in the art of pharmacology. In general, such preparatory methods include the step of combining said active ingredients with a carrier and/or one or more other accessory ingredients, and then, if necessary or desirable, shaping or packaging the product into a desired single- or multi-dose unit.

Although the pharmaceutical compositions provided herein are principally directed to those suitable for ethical administration to humans, it is understood that such compositions are generally suitable for mammals of all sorts.

Oral formulations may be prepared, packaged, and/or sold as a discrete solid dose unit such as a tablet, a hard or soft capsule, a cachet, a troche, or a lozenge, each containing a predetermined amount of the active ingredient. Other oral formulations include powdered or granular formulations, aqueous or oily suspensions, aqueous or oily solutions, and emulsions. As used herein, an “oily” liquid is a carbon-containing liquid molecule that exhibits a less polar character than water. Hard capsules and soft gelatin capsules containing an active ingredient may be manufactured using a physiologically degradable composition, such as gelatin. Hard capsules may further include inert solid diluents such as calcium carbonate, calcium phosphate, and kaolin. While calcium diluents are acceptable, its concentration should be minimized to avoid negative interactions with the fatty acids. Soft gelatin capsules containing the MUFAs contemplated herein as the active ingredients may be manufactured using a physiologically degradable composition, such as gelatin. Such soft capsules contain the active ingredient, which may be mixed with other excipients. Liquid pharmaceutical formulations that are suitable for oral administration may be prepared, packaged and sold in liquid form.

Any of the MUFAs, dietary supplement, nutraceuticals, and pharmaceutical compositions described herein may be administered according to the methods described herein. Generally, the compositions described herein may be administered at about 0.01 to about 2 g, about 0.1 to about 1.5 g, or about 0.3 to about 1.3 g per kilogram of body weight, for example, 0.375 g per kg of body weight. The dosage amount may vary according to other factors such as the person's age, health, and/or severity of existing NAFLD or NASH.

By way of non-limiting examples, the compositions described herein may be administered to a person at about 1 gram to about 60 grams per day or any value in between. A person weighing over 72 kg, can be administered doses ranging at about 2 grams to about 60 grams total per day, preferably about 2 grams, about 10 grams, about 20 grams, about 30 grams, about 40 grams, about 50 grams and about 60 grams total per day or any value in between. Persons weighing less than 72 kg may be administered doses of about 1 gram to about 55 grams total per day or any value in between, preferably, about 1 gram, about 5 grams, about 10 grams, about 15 grams, about 20 grams, about 25 grams, about 30 grams, about 35 grams, about 40 grams, about 45 grams, about 50 grams, about 55 grams or any value in between.

A subject may receive the high concentrations of the MUFA compositions contemplated herein as a dietary supplement, nutraceutical, or pharmaceutical in any form described herein including liquid, solid or semi-solid. The subject may undergo treatment with said high concentrations of the MUFAs compositions before or after the liver disease, for example, fatty acid liver disease such as NAFLD or NASH, are detected. It may be advantageous to administer the MUFAs compositions to subjects having high risk factors for developing liver disease, for example, fatty acid liver disease such as NAFLD or NASH. The methods contemplated herein may be administered daily and continue for about at least about 4, 5, 6, 7, 8, 9 or 10 weeks, until the liver disease, for example, fatty acid liver disease such as NAFLD or NASH disappears, or even longer.

According to some embodiments, the present invention is directed to a method of treating or preventing liver disease in a subject. It may be advantageous to administer the present compositions to subjects having histologic biomarkers that indicate progression, grading or staging of liver disease, for example, fatty acid liver disease, to treat or prevent liver disease. Hepatic biomarkers may include liver-to-body weight ratio (mg/gm), whole blood glucose (mg/dL), plasma alanine aminotransferase (ALT) (U/L), liver hydroxyproline (μg/mg total protein), liver triglyceride (mg/g liver), steatosis score, lobular inflammation score, hepatocyte ballooning score, or fibrosis area (Sirius Red-Positive Area %). Steatosis score, lobular inflammation score, and hepatocyte ballooning score are defined as the NAS components that determine NAFLD Activity Score. Hepatic biomarkers may further comprise NAFLD Activity Score comprising steatosis score, lobular inflammation score, and hepatocyte ballooning score. Determinants of the steatosis, inflammation and ballooning scores are as known in the art, for example in Kleiner D E, Brunt E M, Van Natta M, Behling C, Contos M J, Cummings O W, et al. Design and validation of a histological scoring system for nonalcoholic fatty liver disease. Hepatology 2005; 41:1313-1321, and as briefly described herein. Such biomarkers can be determined via a blood sample and/or liver biopsy by methods known in the art and as described briefly herein.

According to some embodiments, the methods of the present invention further include identifying a subject suitable for treatment with the compositions described herein. For example, the subject may be identified as suffering from liver disease, e.g., NASH or NAFLD. In another embodiment, the methods of the present invention further include assessing the progression of disease following treatment. Methods of assessing whether a subject is suitable for treatment with the compositions disclosed herein or for assessing the progression of disease can involve assessing the levels of biomarkers as set forth above. For example, the method comprises extracting at least one biological sample from a subject and analyzing the sample for one or more hepatic biomarkers. Extraction of biological samples can comprise drawing whole blood sample from a potentially at-risk subject, obtaining a liver biopsy from a potentially at-risk subject, or both. Examples of biopsies include a punch biopsy, a needle biopsy, or a laparoscopic clamshell biopsy. According to some embodiments, analyzing the biological sample for one or more hepatic biomarkers comprises establishing the existence or amount of the hepatic biomarker, comparing it to the appropriate baseline parameter of healthy levels or unhealthy levels of said biomarker or from the subject at an earlier time point, e.g., before treatment.

According to some embodiments, the methods contemplated herein treat or prevent liver disease in a subject, improve the metabolic functioning of the liver of a subject, or both. According to some embodiments, the methods contemplated herein treat or prevent liver disease in a subject. According to some embodiments, the treatment or prevention of liver disease in a subject comprises the modulation of one or more hepatic biomarkers. According to some embodiments, the methods contemplated herein improve the metabolic functioning of the liver of a subject. According to some embodiments, liver function improvements comprise modulation of one or more hepatic biomarkers.

According to some embodiments, the methods contemplated herein comprise the modulation of one or more hepatic biomarkers. According to some embodiments, hepatic biomarkers comprise at least one of liver-to-body weight ratio (mg/gm), whole blood glucose (mg/dL), plasma alanine aminotransferase (ALT) (U/L), liver hydroxyproline (μg/mg total protein), liver triglyceride (mg/g liver), steatosis score, lobular inflammation score, hepatocyte ballooning score, NAFLD Activity Score, or fibrosis area (Sirius Red-Positive Area %), all of the foregoing, or any combination thereof. In one embodiment, liver function improvements comprise modulation of liver-to-body weight ratio (mg/gm), whole blood glucose (mg/dL), plasma alanine aminotransferase (ALT) (U/L), liver hydroxyproline (μg/mg total protein), hepatocyte ballooning score, NAFLD Activity Score, or fibrosis area (Sirius Red-Positive Area %), all of the foregoing, or any combination thereof.

In one embodiment, modulation of hepatic biomarkers comprises reduction of the biomarker thereof when compared to a control. In one embodiment, liver-to-body weight ratio (mg/gm) is reduced by about 0.1% to about 15.0%, about 5.0% to about 10%, or about 5.5% to about 9.5% compared to a control. In one embodiment, whole blood glucose (mg/dL) is reduced by about 0.1% to about 20.0%, about 8.0% to about 14.0%, or about 10.5% to about 11.3% compared to a control. In one embodiment, plasma alanine aminotransferase (ALT) (U/L), liver hydroxyproline (μg/mg total protein) is reduced by about 0.1% to about 30.0%, about 10.0% to about 20.0%, or about 18.0% to about 19.6% compared to a control. In one embodiment, hepatocyte ballooning score is reduced by a factor of 1 compared to a control. In one embodiment, NAFLD Activity Score or the NASH Activity Score is reduced by about 0.1% to about 15.0%, about 5.0% to about 10.0%, or about 9.0 to about 9.3% compared to a control. In one embodiment, fibrosis area (Sirius Red-Positive Area %) is reduced by about 0.1% to about 50.0%, about 10.0% to about 40.0%, or about 23.0% to about 33.0% compared to a control.

The MUFAs composition comprises a combination, mixture or blend of a high concentration of MUFAs having melting point temperatures of about −1.7° C. to about 10.7° C. and iodine values of about 50 to about 130. The said high concentration of MUFAs may contribute to at least about 80% to about 100% by weight of the total composition. The said MUFAs may further contribute to at least about 90% to about 95% by weight of the total composition. Optionally, other fatty acids may be incorporated into the said high MUFAs composition. Such optional fatty acids may be saturated and polyunsaturated fatty acids. It is preferred that such optional fatty acids comprise no more than 15% of the total weight of the composition.

Preferred fatty acids from the short carbon chain group include C16:1 and from the long carbon chain group include C18:1. The compositions described herein are not limited to a single fatty acid selected from each group. For example, C16:1 is selected as a representative MUFA, however, it may be combined with other short carbon chain members of the group including C12:1 or C14:1 such that the entire composition of the short chain MUFA members may not exceed 40% by weight of the composition. Likewise, the long carbon chain members of the MUFA discussed herein are not limited to C18:1 as it may be comprised of other members including C22:1 and C20:1 such that the entire composition of the long carbon chain members do not exceed about 80%. The combination of short carbon chain and long carbon chain MUFAs may comprise at least about 80% to about 100% by weight of the total composition. The combined short carbon chain and long carbon chain MUFAs may further comprise at least about 90% to about 95% by weight of the total composition.

Optionally, other fatty acids including saturated and polyunsaturated fatty acids may be incorporated into the C16:1 and C18:1 fatty acid composition. Such optional saturated fatty acid and polyunsaturated fatty acids may not exceed more than about 15% by weight of the entire composition. The saturated fatty acids may comprise a single or multiple members of saturated fatty acids and may further comprise any value up to about 15% by weight of the total composition, preferably up to about 7% by weight, and more preferably about 1% to about 5% by weight of the total composition. Non-limiting examples of saturated fatty acids include C16:0, C12:0, C14:0, and C10:0. TABLE 5 provides common saturated fatty acids contemplated in the present invention.

TABLE 5
Saturated Fatty Acid

| Structure | Name |
|---|---|
| C4:0 | Butyric |
| C6:0 | Caproic |
| C8:0 | Caprylic |
| C10:0 | Capric |
| C12:0 | Lauric |
| C14:0 | Myristic |
| C16:0 | Palmitic |
| C18:0 | Steric |
| C20:0 | Arachidic |
| C22:0 | Behenic |
| C24:0 | Lignoceric |

The polyunsaturated fatty acids may comprises a single or multiple members of polyunsaturated fatty acids and may further comprise any value up to about 15% by weight of the total composition, preferably up to about 7% by weight, and more preferably about 1% to about 5% by weight of the total composition. Non-limiting examples of polyunsaturated fatty acids include C18:2, C18:3, C20:4, C20:5 and C22:6. TABLE 6 provides common polyunsaturated fatty acids contemplated in the present invention.

TABLE 6
Polyunsaturated Fatty Acids

| Structure | Name |
|---|---|
| C16:2 | Hexadecadienoic |
| C16:4 | Hexadecatetradienoic |
| C18:2 | Linoleic |
| C18:3 | Linolenic |
| C20:4 | Arachidonic |
| C20:5 | Eicosapentaenoic |
| C21:5 | Heneicosanoic |
| C22:2 | Docosadienoic |
| C22:3 | Docosatrienoic |
| C22:4 | Docosatetraenoic |
| C22:5 | Docosapentaenoic acid (DPA) |
| C22:6 | Docosahexaenoic acid (DHA) |

Optionally, the composition may comprise trace or minute amounts of other fatty acids. The trace fatty acids will be understood to refer to any fatty acids found commonly in the mammal's diet ranging from C8:0 to (C22:6) including those fatty acids described herein. Such trace amounts of fatty acids may optionally be present at up to about 3% by weight of the total composition.

The MUFA discussed herein including C16:1 and C18:1 is typically formulated, meaning that the individual components are first refined and purified from its source material, then mixed together to form the desired composition described herein. It is further contemplated that naturally occurring foodstuffs can be enhanced into the composition described herein by adding, adjusting or substituting the foodstuff with at least one of the MUFA described herein such that the desired percentage weight of the C16:1 and C18:1 and the desired melting point range, as set forth herein, is reached. Other fatty acids including saturated fatty acids, and polyunsaturated fatty acids may optionally be present in said enhanced composition, however, preferably such optional fatty acids may not exceed about 15% by weight of the overall composition.

The term “melting point” refers to the melting point of a mixture of a first monounsaturated fatty acid and a second monounsaturated fatty acid and, if present, additional monounsaturated fatty acids that may be present.

Exemplary compositions including C16:1 and C18:1, and their melting points are provided in TABLE 7:

TABLE 7

| | | | Melting |
|---|---|---|---|
| C16:1 | C18:1 | | point |
| (mg) | (mg) | % C16:1 | (° C.) |
| 23 | 82 | 22.1 | 7.94 |
| 30 | 70 | 30.2 | 6.6 |
| 41 | 61 | 40.4 | 3.29 |

Further representative compositions encompassed by the present invention and within the scope of the invention are below. These examples and preparations which follow are provided to enable those skilled in the art to more clearly understand and to practice the present invention. They should not be considered as limiting the scope of the invention, but merely as being illustrative and representative thereof.

Composition I

| | |
|---|---|
| 18-23% | C16:1 |
| 60-80% | C18:1 |
| 0-5% | saturated fatty acid |
| 0-5% | polyunsaturated fatty acid |
| 0-3%. | trace fatty acids |

Composition II

| | |
|---|---|
| 18-23% | C16:1 |
| 60-80% | C18:1 |
| 2-5% | C16:0 |
| 2-5% | C18:2 |
| 1-3%. | trace fatty acids |

Composition III

| | |
|---|---|
| 20% | C16:1 |
| 60%-80% | C18:1 |
| 2%-5% | C16:0 |
| 2%-5% | C18:2 |
| 1%-3% | trace fatty acids |

Composition IV

| | |
|---|---|
| 20%-40% | C16:1 |
| 40%-60% | C18:1 |
| 0-5% | saturated fatty acid |
| 0-5% | polyunsaturated fatty acid |
| 0-3% | trace fatty acids |

Composition V

| | |
|---|---|
| 20%-23% | C16:1 |
| 60%-80% | C18:1 |
| 1%-5% | saturated fatty acid |
| 1%-5% | polyunsaturated fatty acid |

Composition VI

| | |
|---|---|
| 20%-40% | C16:1 |
| 40%-60% | C18:1 |
| 0%-5% | saturated fatty acid |
| 0%-5% | polyunsaturated fatty acid |

Composition VII

| | |
|---|---|
| 20%-40% | C16:1, C14:1 and C12:1 |
| 40%-80% | C22:1, C20:1, and C18:1 |
| Up to 15% | saturated fatty acid and |
| | polyunsaturated fatty acid |

Composition VIII

| | |
|---|---|
| 20% | C16:1 |
| 60% | C18:1 |
| 15% | C16:0 |
| 5% | C18:2 |

In one embodiment, the compositions disclosed herein above may be diluted with or co-administered with carrier fatty acids. In one embodiment, the carrier fatty acid is selected from the group consisting of safflower oil, olive oil, palm oil, soybean oil, canola oil (rapeseed oil), corn oil, peanut oil, other vegetable oils, and a combination thereof.

As used herein, the term “about” means plus or minus 20% of the numerical value of the number with which it is being used. Therefore, about 50% means in the range of 40%-60%.

In order that the invention described herein may be more fully understood, the following examples are set forth. It should be understood that these examples are for illustrative purposes only and are not to be construed as limiting this invention in any manner.

All features of each of the aspects of the invention apply to all other aspects mutatis mutandis. The contents of all references, patents, pending patent applications and published patents, cited throughout this application are hereby expressly incorporated by reference.

### Example 1: Effects of Composition A in STAM Model of Non-Alcoholic Steatohepatitis

### 1. Study Objective

To examine the effects of Composition A in STAM model of non-alcoholic steatohepatitis. Composition A included the following elements:

Composition A

| | |
|---|---|
| 20% | C16:1n7 |
| 70% | C18:1 |
| 5% | C16:0 |
| 5% | C18:2 |

### 2. Materials and Methods

### 2.1. Test Substances

To prepare dosing solution, Composition A was diluted with safflower oil (Lot #; T-18502, CLEA Japan Inc., Japan). Experimental diet was prepared by high-fat diet without safflower oil (Lot #; T-18480, CLEA Japan Inc.).

### 2.2. Induction of NASH

NASH was induced in 16 male mice by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Inc.) from 4 to 7 weeks of age.

### 2.3. Route of Drug Administration

Composition A was administered orally in a volume of 200 μL/mouse.

### 2.4. Treatment Doses

Composition A was administered at a dose of 100% three times daily from Day 0 to Day 2 (on 7 weeks of age).

Composition A was administered at 2 dose levels of 50% and 25% three times daily from Day 3 to Day 13 (from 7 to 9 weeks of age).

### 2.5. Animals

C57BL/6 mice (14-day-pregnant female) were obtained from Japan SLC, Inc (Japan). All animals used in the study were housed and cared for in accordance with the Japanese Pharmacological Society Guidelines for Animal Use.

### 2.5.1. Environment

The animals were maintained in a SPF facility under controlled conditions of temperature (23±2° C.), humidity (45±10%), lighting (12-hour artificial light and dark cycles; light from 8:00 to 20:00) and air exchange. A high pressure was maintained in the experimental room to prevent contamination of the facility.

### 2.5.2. Animal Husbandry

The animals were housed in TPX cages (CLEA Japan Inc.) with a maximum of 4 mice per cage. Sterilized Paper-Clean (Japan SLC) was used for bedding and replaced once a week.

### 2.5.3. Food and Drink

Sterilized powdered safflower oil-free HFD was provided ad libitum, being placed in powder food feeder (Cat #; CL-0932, CLEA Japan Inc.) on the bottom of the cage. Pure water was provided ad libitum from a water bottle equipped with a rubber stopper and a sipper tube. Water bottles were replaced once a week, cleaned, and sterilized in an autoclave and reused.

### 2.5.4. Animal and Cage Identification

Mice were identified by ear punch. Each cage was labeled with a specific identification code.

### 2.6. Measurement of Food Intake

Food consumption was measured once daily per cage during the treatment period. Food ration in Group 1 from Day 2 to Day 14 was restricted based on the average of food consumption in mice of Composition A group at Day 1.

### 2.7. Measurement of Whole Blood and Plasma Biochemistry

Non-fasting blood glucose was measured in whole blood using Stat Strip glucose meter (NIPRO CORPORATION, Japan). For plasma biochemistry, non-fasting blood was collected in polypropylene tubes with anticoagulant (Novo-Heparin, Mochida Pharmaceutical Co. Ltd., Japan) and centrifuged at 1,000×g for 15 minutes at 4° C. The supernatant was collected and stored at −80° C. until use. Plasma ALT level was measured by FUJI DRI-CHEM 7000 (Fujifilm, Japan).

### 2.8. Measurement of Liver Biochemistry

To quantify liver hydroxyproline content, frozen liver samples were processed by an alkaline-acid hydrolysis method as follows. Liver samples were defatted with 100% acetone, dried in the air, dissolved in 2N NaOH at 65° C., and autoclaved at 121° C. for 20 minutes. The lysed samples (400 μL) were acid-hydrolyzed with 400 μL of 6N HCl at 121° C. for 20 minutes, and neutralized with 400 μL of 4N NaOH containing 10 mg/mL activated carbon. AC buffer (2.2M acetic acid/0.48M citric acid, 400 μL) was added to the samples, followed by centrifugation to collect the supernatant. A standard curve of hydroxyproline was constructed with serial dilutions of trans-4-hydroxy-L-proline (Sigma-Aldrich) starting at 16 μg/mL. The prepared samples and standards (each 400 μL) were mixed with 400 μL chloramine T solution (Wako Pure Chemical Industries) and incubated for 25 minutes at room temperature. The samples were then mixed with Ehrlich's solution (400 μL) and heated at 65° C. for 20 minutes to develop the color. After samples were cooled on ice and centrifuged to remove precipitates, the optical density of each supernatant was measured at 560 nm. The concentrations of hydroxyproline were calculated from the hydroxyproline standard curve. Protein concentrations of liver samples were determined using a BCA protein assay kit (Thermo Fisher Scientific, USA) and used to normalize the calculated hydroxyproline values. Liver hydroxyproline levels were expressed as μg per mg protein.

To quantify liver triglyceride content, liver total lipid-extracts were obtained by Folch's method (Folch J. et al., J. Biol. Chem. 1957; 226: 497). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride content was measured by Triglyceride E-test (FUJIFILM Wak Pure Chemical Corporation, Japan).

### 2.9. Measurement of Palmitoleic Acid (C16:1)

For determination of palmitoleic acid concentration in the mouse brain, whole mouse brain was collected and stored at −80° C. Mouse brain palmitoleic acid (C16:1) level was quantified by LC-MS/MS at Lipidome lab Inc. (Japan).

### 2.10. Histological Analyses

For HE staining, sections were cut from paraffin blocks of liver tissue prefixed in Bouin's solution and stained with Lillie-Mayer's Hematoxylin (Muto Pure Chemicals Co., Ltd., Japan) and eosin solution (FUJIFILM Wako Pure Chemical Corporation, Japan). NAFLD Activity score (NAS) was calculated according to the criteria of Kleiner (Kleiner D E. et al., Hepatology, 2005; 41:1313).

To visualize collagen deposition, Bouin's fixed liver sections were stained using picro-Sirius red solution (Waldeck, Germany). For quantitative analysis of fibrosis areas, bright field images of Sirius red-stained sections were captured around the central vein using a digital camera (DFC295; Leica, Germany) at 200-fold magnification, and the positive areas in 5 fields/section were measured using ImageJ software (National Institute of Health, USA).

### 2.11. Sample Collection

For plasma samples, non-fasting blood was collected in polypropylene tubes with anticoagulant (Novo-Heparin) and centrifuged at 1,000×g for 15 minutes at 4° C. The 20 μL of supernatant was collected and stored at −80° C. for biochemistry. The remaining plasma was collected and stored at −80° C. for shipping.

For liver samples, left lateral lobe and caudate lobe were collected and snap frozen in liquid nitrogen and stored at −80° C. for shipping. Left and right medial lobe were snap frozen in liquid nitrogen and stored at −80° C. for liver biochemistry. Right lobe was fixed in Bouin's solution and then embedded in paraffin. Samples were stored at room temperature for histology.

For brain samples, whole brain at the mice IDs of 101, 106 and 108 were collected and snap frozen in liquid nitrogen. Samples were stored at −80° C. for brain biochemistry.

### 2.12. Statistical Tests

Results were expressed as mean±SD.

### 3. Experimental Design and Treatment

### 3.1. Study Groups

At Day 0-Day 2 (7 weeks of age)

### Group 1: Control

Eight NASH mice were orally administered safflower oil in a volume of 200 μL/mouse three times daily (total of 600 μL/mouse/day) and were fed with powdered HFD (without safflower oil) using powder food feeder from Day 0 to Day 2.

### Group 2: Composition A

Eight NASH mice were orally administered Composition A in a volume of 200 μL/mouse three times daily (total of 600 μL/mouse/day) and were fed with powdered HFD (without safflower oil) using powder food feeder from Day 0 to Day 2.

The table below summarizes the treatment schedule

| | No. | | Test | Volume | | |
|---|---|---|---|---|---|---|
| Group | mice | Mice | substance | (μL/mouse) | Regimen | Sacrifice |
| 1 | 8 | STAM | Safflower oil | 200 | Safflower | — |
| | | | | | oil-free HFD, | |
| | | | | | PO, TID, | |
| | | | | | Day 0-2 | |
| 2 | 8 | STAM | Composition A | 200 | Safflower | — |
| | | | | | oil-free HFD, | |
| | | | | | PO, TID, | |
| | | | | | Day 0-2 | |

At Day 3-Day 14 (7 to 9 weeks of age)

### Group 1: Control

Four NASH mice were orally administered safflower oil in a volume of 200 μL/mouse three times daily (total of 600 μL/mouse/day) and were fed with powdered HFD (without safflower oil) using powder food feeder from Day 3 to Day 13.

### Group 2: 50% Composition/50% Safflower Oil

Two NASH mice were orally administered 50% Composition/50% safflower oil in a volume of 200 μL/mouse three times daily (total of 600 μL/mouse/day) and were fed with powdered HFD (without safflower oil) using powder food feeder from Day 3 to Day 13.

### Group 3: 25% Composition/75% Safflower Oil

Two NASH mice were orally administered 25% Composition/75% safflower oil in a volume of 200 μL/mouse three times daily (total of 600 μL/mouse/day) and were fed with powdered HFD (without safflower oil) using powder food feeder from Day 3 to Day 13.

The table below summarizes the treatment schedule.

| | No. | | Test | Volume | | |
|---|---|---|---|---|---|---|
| Group | mice | Mice | substance | (μL/mouse) | Regimen | Sacrifice |
| 1 | 4 | STAM | Safflower oil | 200 | Safflower | Day 14 |
| | | | | | oil-free HFD, | |
| | | | | | PO, TID, | |
| | | | | | Day 3-13 | |
| 2 | 2 | STAM | 50% | 200 | Safflower | Day 14 |
| | | | Composition | | oil-free HFD, | |
| | | | A/50% | | PO, TID, | |
| | | | safflower oil | | Day 3-13 | |
| 3 | 2 | STAM | 25% | 200 | Safflower | Day 14 |
| | | | Composition | | oil-free HFD, | |
| | | | A/75% | | PO, TID, | |
| | | | safflower oil | | Day 3-13 | |

### 3.2. Animal Monitoring and Sacrifice

The viability, clinical signs and behavior were monitored daily. Body weight was recorded before the treatment. Mice were observed for significant clinical signs of toxicity, moribundity and mortality approximately 60 minutes after each administration. Because animals showed significant clinical signs of moribundity, the animals were euthanized ahead of study termination. The samples were collected from dead or euthanized animals. The animals were sacrificed at 9 weeks of age (Day 14) by exsanguination through direct cardiac puncture under isoflurane anesthesia (Pfizer Inc.).

### 4. Results

### 4.1. Body Weight Changes and General Condition

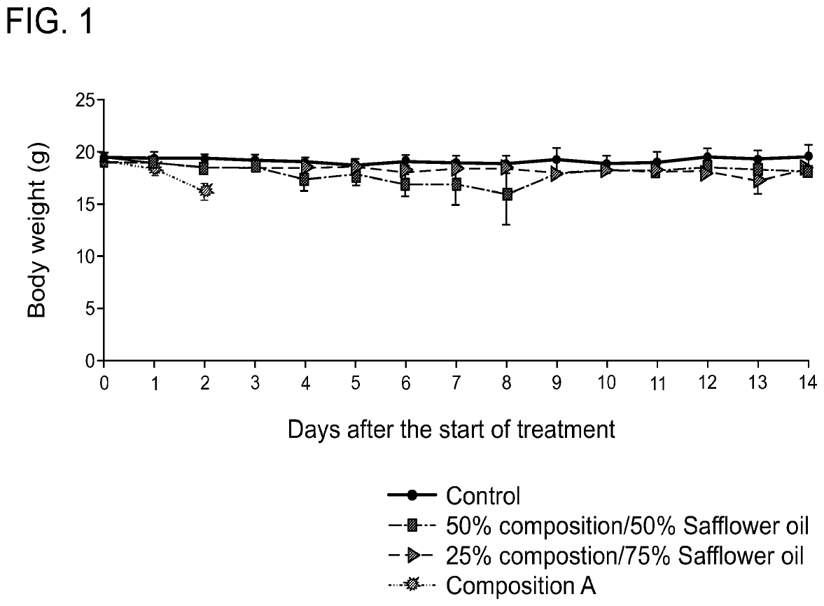

### 4.1.1. Body Weight Changes (FIG. 1)

As all mice of the Composition A group showed death or moribundity by Day 2, the mice of Control group were rearranged to 50% Composition/50% Safflower oil and 25% Composition/75% Safflower oil groups.

During the treatment period, mice found dead before reaching Day 14 were as follows; four out of 8 mice were found dead and four out of 8 mice were euthanized in the Composition A group. One out of 2 mice was euthanized in the 50% Composition/50% Safflower oil and 25% Composition/75% Safflower oil groups.

### 4.2. Food Intake

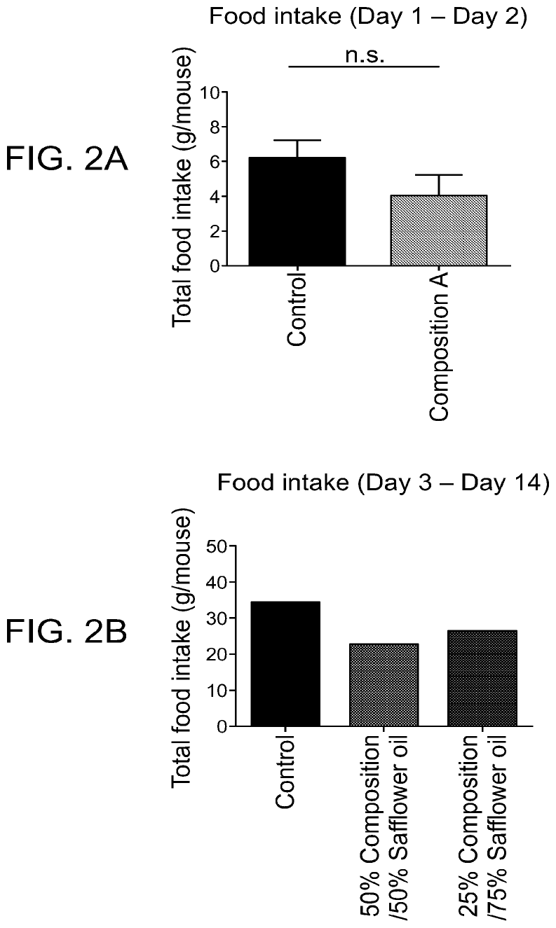

Food intake is shown in FIG. 2A and FIG. 2B.

### 4.3. Body Weight on the Day of Sacrifice and Liver Weight

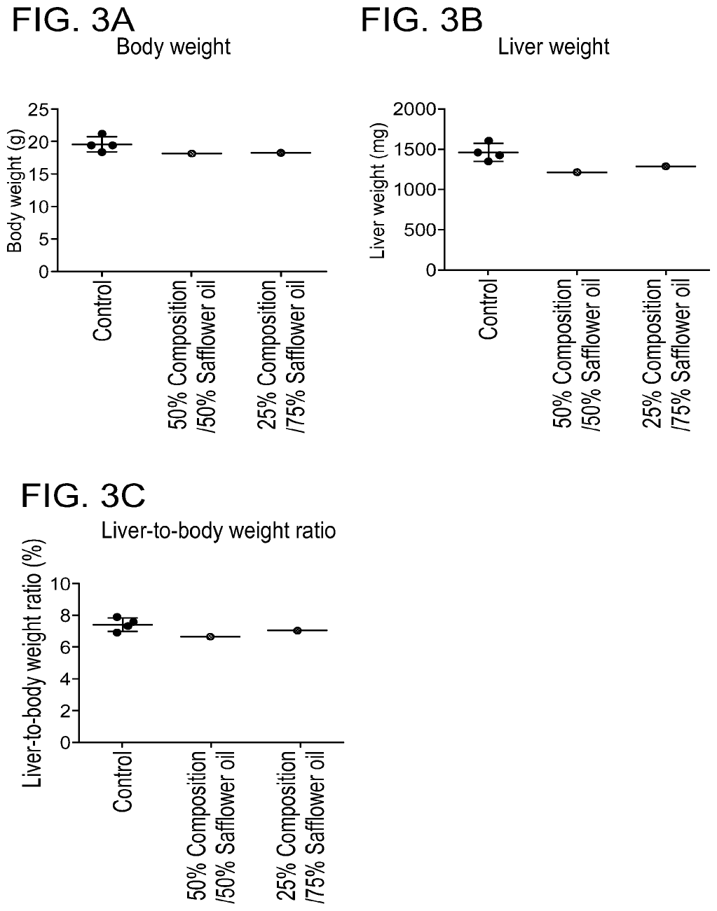

### 4.3.1. Body Weight on the Day of Sacrifice (FIG. 3A and Table A)

Mean body weight on the day of sacrifice is shown in FIG. 3A.

### 4.3.2. Liver Weight and Liver-to-Body Weight Ratio (FIGS. 3B and 3C and Table A)

Mean liver weigh and mean liver-to-body weight ratio are shown in FIGS. 3B and 3C.

TABLE A
Body weight and liver weight

| | | 50% Composition/ | 25% Composition/ |
|---|---|---|---|
| Parameter | Control | 50% Safflower | 75% Safflower |
| (Mean ± SD) | (n = 4) | oil (n = 1) | oil (n = 1) |
| Body | 19.6 ± 1.2 | 18.2 | 18.3 |
| weight (g) | | | |
| Liver | 1460 ± 111 | 1216 | 1284 |
| weight (mg) | | | |
| Liver-to-body | 7.4 ± 0.4 | 6.7 | 7 |
| weight | | | |
| ratio (%) | | | |

### 4.4. Biochemistry

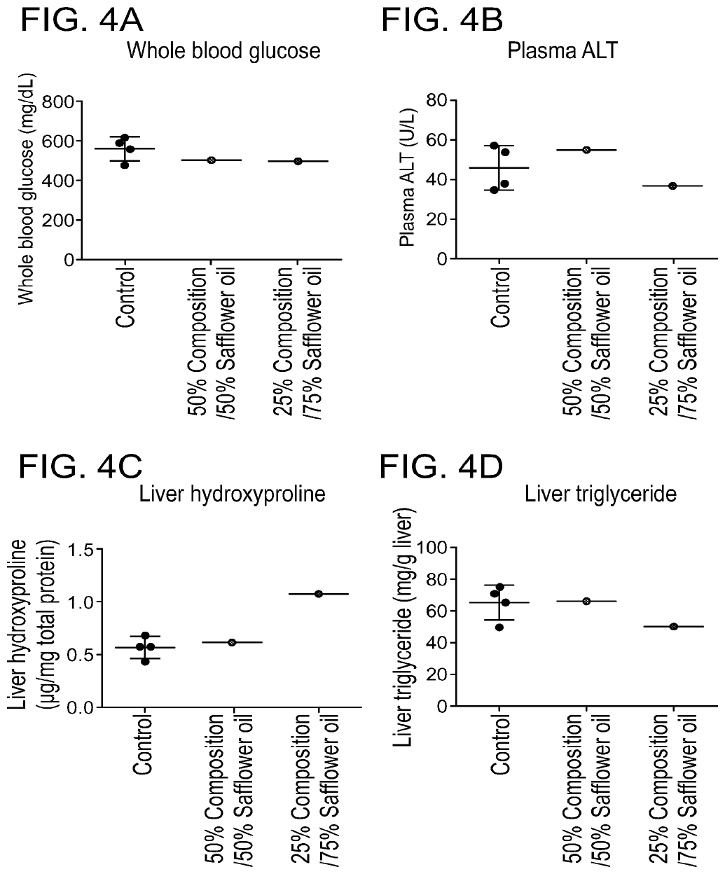

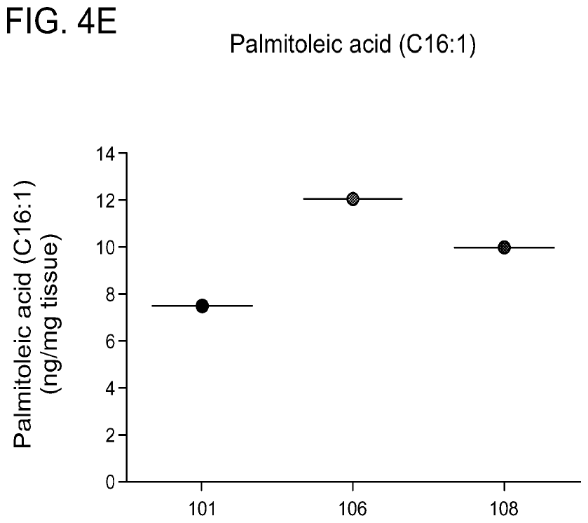

### 4.4.1. Whole Blood Glucose (FIG. 4A and Table B)

Whole blood glucose levels are shown in FIG. 4A.

### 4.4.2. Plasma ALT (FIG. 4B and Table B)

Plasma ALT levels are shown in FIG. 4B.

### 4.4.3. Liver Hydroxyproline (FIG. 4C and Table B)

Liver hydroxyproline contents are shown in FIG. 4C.

### 4.4.4. Liver Triglyceride (FIG. 4D and Table B)

Liver triglyceride contents are shown in FIG. 4D.

TABLE B
Biochemistry

| | | 50% Composition/ | 25% Composition/ |
|---|---|---|---|
| Parameter | Control | 50% Safflower | 75% Safflower |
| (Mean ± SD) | (n = 4) | oil (n = 1) | oil (n = 1) |
| Whole blood | 563 ± 60 | 504 | 500 |
| glucose | | | |
| (mg/dL) | | | |
| Plasma | 46 ± 11 | 55 | 37 |
| ALT (U/L) | | | |
| Liver | 65.2 ± 11.0 | 66.2 | 50.2 |
| hydroxyproline | | | |
| (μg/mg | | | |
| total protein) | | | |
| Liver | 0.6 ± 0.1 | 0.6 | 1.1 |
| triglyceride | | | |
| (mg/g liver) | | | |

### 4.4.5. Palmitoleic Acid (C16:1) (FIG. 4E and Table C)

Palmitoleic acid levels are shown in FIG. 4E.

TABLE C
Palmitoleic acid

| Parameter | 102 | 106 | 108 |
|---|---|---|---|
| Palmitoleic acid (C16:1) ng/mg | 7.53 | 12.1 | 10.01 |
| tissue | | | |

### 4.5. Histological Analyses

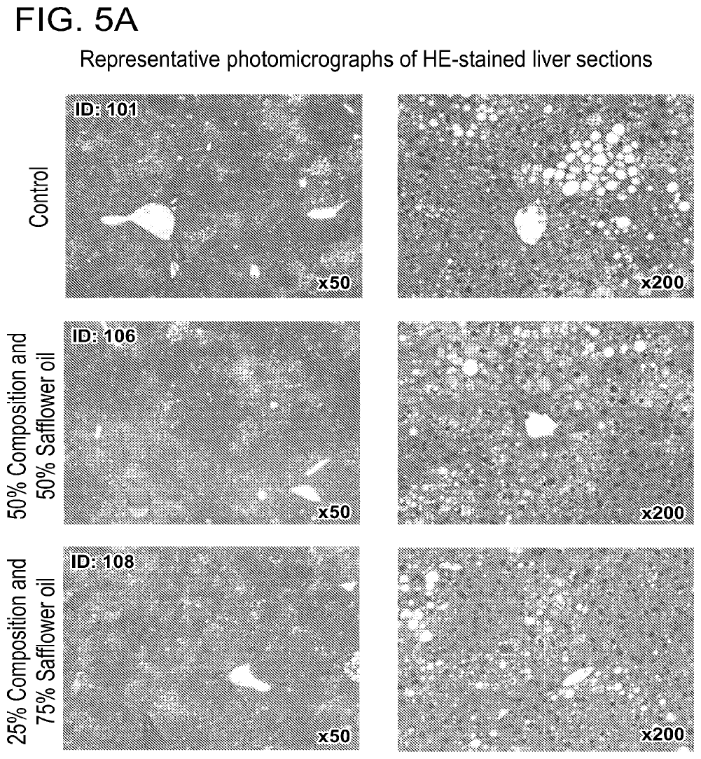

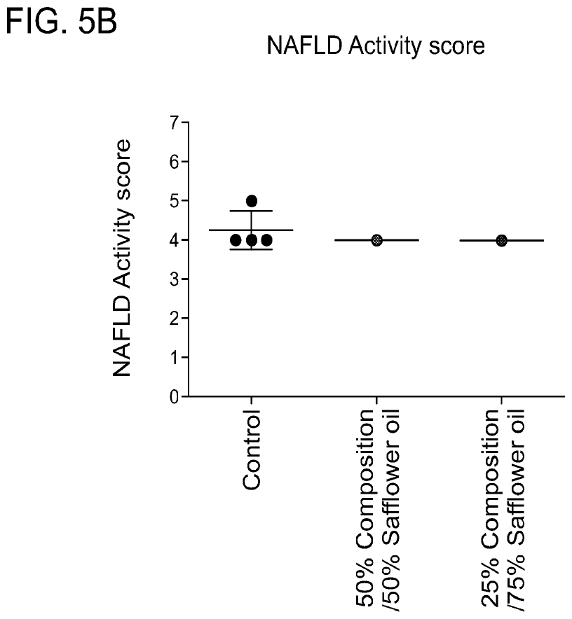

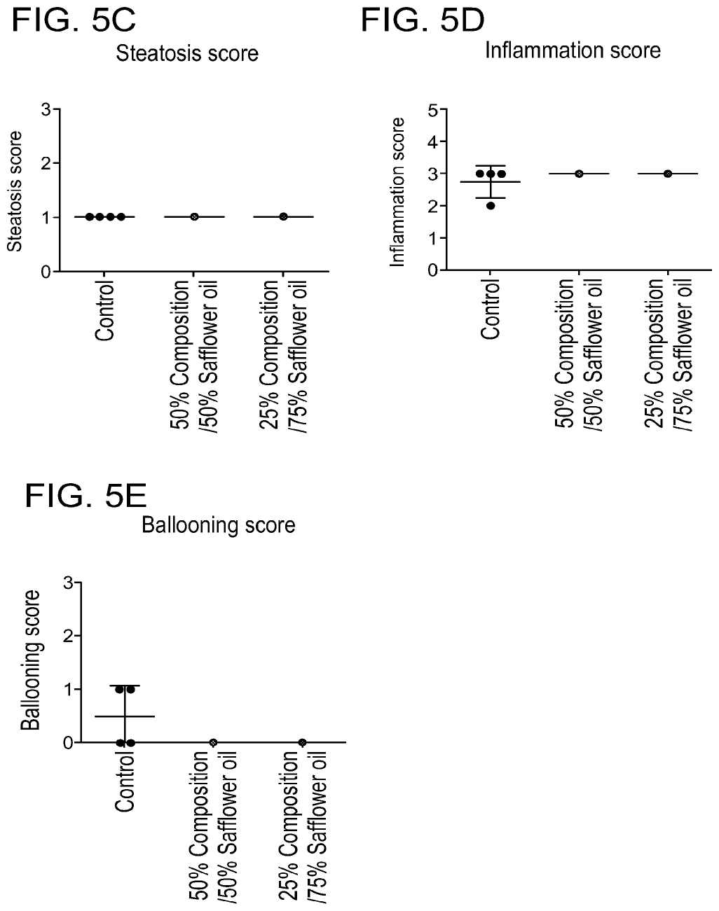

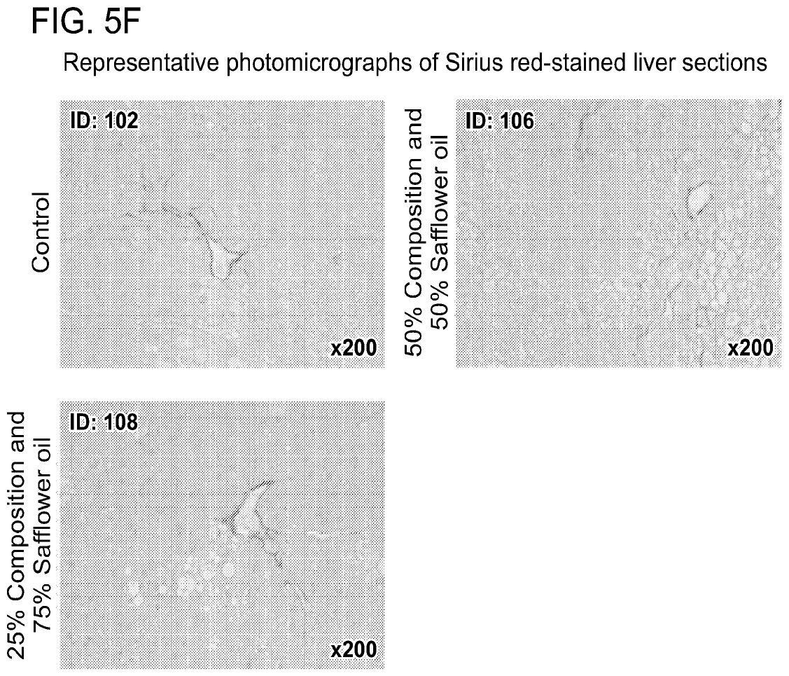

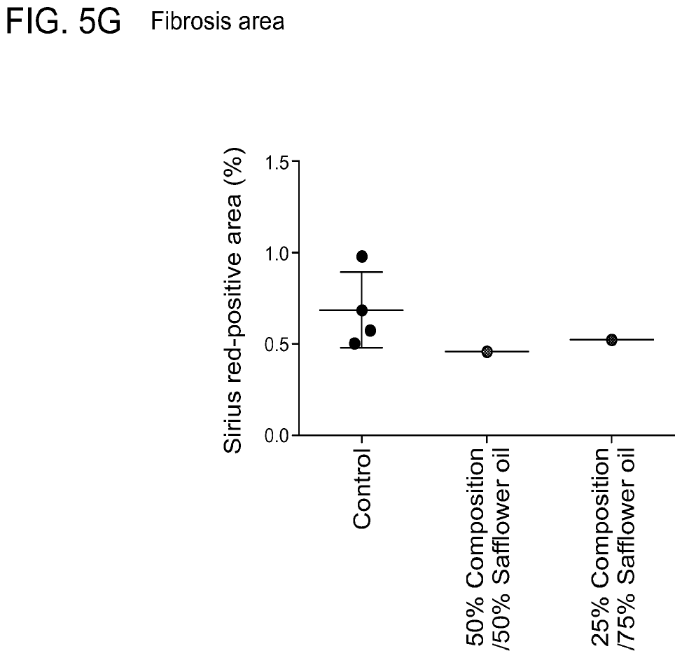

4.5.1. HE staining and NAFLD Activity score (FIG. 5A, 5B, 5C, 5D AND 5E; and Table D)

Representative photomicrographs of HE-stained liver sections and NAFLD activity score are shown in FIGS. 5A and 5B.

TABLE D
NAFLD Activity score

| | Score | | | | | | | | | | | |
|---|---|---|---|---|---|---|---|---|---|---|---|---|
| | | | | | Lobular | | | | Hepatocyte | | | |
| | Steatosis | | | | inflammation | | | | ballooning | | | NAS |
| Group | 0 | 1 | 2 | 3 | 0 | 1 | 2 | 3 | 0 | 1 | 2 | (mean ± SD) |
| Control | — | 4 | — | — | — | — | 1 | 3 | 2 | 2 | — | 4.3 ± 0.5 |
| (n = 4) | | | | | | | | | | | | |
| 50% | — | 1 | — | — | — | — | — | 1 | 1 | — | — | 4 |
| Composition/ | | | | | | | | | | | | |
| 50% | | | | | | | | | | | | |
| Safflower oil | | | | | | | | | | | | |
| (n = 1) | | | | | | | | | | | | |
| 25% | — | 1 | — | — | — | — | — | 1 | 1 | — | — | 4 |
| Composition/ | | | | | | | | | | | | |
| 75% | | | | | | | | | | | | |
| Safflower oil | | | | | | | | | | | | |
| (n = 1) | | | | | | | | | | | | |

### 4.5.2. Sirius Red Staining and the Fibrosis Area (FIGS. 5F and 5G and Table E)

Representative photomicrographs of Sirius red-stained liver sections and the fibrosis area (Sirius red-positive area) are shown in FIGS. 5F and 5G.

TABLE E
Fibrosis area

| | | 50% Composition/ | 25% Composition/ |
|---|---|---|---|
| Parameter | Control | 50% Safflower oil | 75% Safflower oil |
| (Mean ± SD) | (n = 4) | (n = 1) | (n = 1) |
| Sirius | 0.69 + 0.21 | 0.46 | 0.53 |
| red-positive | | | |
| area (%) | | | |

TABLE F
Definition of NAS Components

| Item | Score | Extent |
|---|---|---|
| Steatosis | 0 | <5% |
| | 1 | 5-33% |
| | 2 | >33-66% |
| | 3 | >66% |
| Lobular | 0 | No foci |
| Inflammation | 1 | <2 foci/200x |
| | 2 | 2-4 foci/200x |
| | 3 | >4 foci/200x |
| Hepatocyte | 0 | None |
| Ballooning | 1 | Few balloon cells |
| | 2 | Many cells/prominent ballooning |

### 5. Conclusions

The results show that fatty deposits were evident upon microscopic examination of liver tissue from animals administered high fat or saturated fat dietary compositions, but surprisingly were absent in animals administered an equally high fat dietary composition containing elevated amounts of short chain MUFA triglycerides.

Specifically, neither the 50/50 Group nor the 25/75 Group showed any Hepatocyte Ballooning, whereas 50% of Control Group did, resulting in a 9.3% reduction in NAFLD Activity Score compared to Control Group.

In addition, Fibrosis Area (Sirius Red-Positive area %) was reduced in treatment groups:
Control Group 0.69+/−0.21
50/50 Group: 0.46, 33% reduction from Control
25/75 Group: 0.53, 23% reduction from Control.

Liver-to-body weight ratio (mg/gm) was reduced in treatment groups:
Control Group: 7.4+/−0.4
50/50 Group: 6.7, 9.5% reduction from Control
25/75 Group: 7.0, 5.5% reduction from Control.

Whole Blood Glucose (mg/dL) was reduced in both Treatment Groups:
Control Group: 563+/−60
50/50 Group: 504, 10.5% reduction from Control
25/75 Group: 500, 11.3% reduction from Control.

Plasma ALT (U/L) is an indicator of liver damage:
Control Group: 46+/−11
50/50 Group: 55, 19.5% increase from Control
25/75 Group: 37, 19.6% reduction from Control.

Liver Hydroxyproline (μg/mg) is a marker of liver fibrosis:
Control Group: 65.2+/−11.0
50/50 Group: 66.2, 1.5% increase from Control
25/75 Group: 50.2, 23% reduction from Control.

We are of the opinion that the negative animal response may have been an allergic reaction to the composition since it was sourced from macadamia nuts.

When there was a negative animal response, the amount of Safflower was increased to dilute the composition in hopes it would reduce or eliminate any negative response from the animals. Safflower oil was in essence being used as a placebo to make sure that the intake (caloric/weight) from both groups was equal with minimal effect. While some results of 25/75 were better than 50/50, there were others where 50/50 had better results.

### Example 2. Effects of a Composition in CDAA-HFD Model of Progressive Human Fibrotic NASH

### 1. Study Objective

To examine the effects of compositions described herein, for example, Composition A, a dosing solution will be tested in a choline-deficient L-amino acid-defined (CDAA)-high fat diet (HFD) model resembling progressive human fibrotic non-alcoholic steatohepatitis. Composition A may include the following elements:

Composition A

| | |
|---|---|
| 20% | C16:1n7 |
| 70% | C18:1 |
| 5% | C16:0 |
| 5% | C18:2 |

### 2. Materials and Methods

### 2.1. Test Substances

Dosing solution may be prepared as described above or diluted as needed, for example with safflower oil.

Control substances may include a vehicle control, such as saline or mineral oil, icobuturate, and elafibranor.

### 2.2. Induction of NASH

NASH will be induced in 4 groups (n=12) of 10-week old male mice (Male C57BL/6JRj) by feeding with high fat diet (24 kcal % HFD, 32% fructose, 1% Cholesterol, without choline, with 0.1% added methionine diet [A16092001; RD]) for 4 weeks prior to study start.

An additional group (n=10) will be fed regular chow as a negative control.

### 2.3. Route of Administration

Test substances will be administered orally.

### 2.4. Dosing

Test substances will be administered once a day.

### 2.5. Measurement of Body Weight

Body weight will be measured once daily for the entire study period.

### 2.6. Measurement of Food Intake

Food intake will be measured once daily during initial 2 weeks of the study period, followed by once weekly (24 h) during remaining study period.

2.7. Whole Blood and/or Plasma Biochemistry

Terminal whole blood will be collected. For plasma biochemistry, plasma will be separated from the whole blood and alanine aminotransferase (ALT), aspartate aminotransferase (AST), free fatty acid (FFA) levels, triglyceride (TG) and total cholesterol (TC) will be measured.

### 2.8. Liver Analysis

Terminal liver will be removed, frozen and sampled for histological analysis, biochemistry, optionally RNA sequencing, and/or kept frozen for further analysis.

For liver biochemistry, terminal liver will processed for TG, TC, FFA, and hydroxyproline (HP) content.

For histology, terminal liver will be biopsied and analyzed for (1) NAFLD Activity Score (hematoxylin and eosin (HE)) including Fibrosis Stage (Picrosirius Red (PSR)), (2) steatosis (HE), (3) Galectin-3 (Gal-3)(immunohistochemistry (IHC)), (4) Collagen 1a1 (col1a1) (IHC), (5) PSR, and (6) alpha-smooth muscle actin (a-SMA) (IHC).

### 3. Experimental Design and Treatment

### 3.1. Study Groups

The animals will be randomized into 5 groups based on body weight.

### Group 1. Negative Control

Group 1 will be LEAN-CHOW fed and administered a Vehicle control.

### Group 2: Placebo Control

Group 2 will be CDAA-HFD fed and administered a Vehicle control.

### Group 3: Test Group

Group 3 will be CDAA-HFD fed and administered the Dosing Solution, such as Composition A.

### Group 4: Positive Control

Group 4 will be CDAA-HFD fed and administered Icobuturate.

### Group 5: Positive Control

Group 5 will be CDAA-HFD fed and administered Elafibranor.

The study period of the study groups will be a total of 8 weeks of dosing per oral gavage, once a day, as described in the table below.

| Group | | | | No. of | | | Dose | Dose | Dose | |
|---|---|---|---|---|---|---|---|---|---|---|
| No. | Animal | Gender | Strain | Animals | Treatment | Administration | freq. | vol. | conc. | |
| 1 | LEAN- | male | C57BL/6JRj | 10 | Vehicle | Oral | 1x/day | 5 ml/kg | — | |
| | CHOW | | | | | | | | | |
| 2 | CDAA- | male | C57BL/6JRj | 12 | Vehicle | Oral | 1x/day | 5 ml/kg | — | |
| | HF | | | | | | | | | |
| 3 | CDAA- | male | C57BL/6JRj | 12 | Dosing | Oral | 1x/day | 5 ml/kg | TBD | |
| | HF | | | | Solution | | | | | |
| 4 | CDAA- | male | C57BL/6JRj | 12 | Icobuturate | Oral | 1x/day | 5 ml/kg | 56/112 | mg/kg |
| | HF | | | | | | | | | |
| 5 | CDAA- | male | C57BL/6JRj | 12 | Elafibranor | Oral | 1x/day | 5 ml/kg | 30 | mg/kg |
| | HF | | | | | | | | | |

### 3.2 Treatment Schedule

Study timeline is tentatively as described in the table below.

| Milestone | Time | Comments |
|---|---|---|
| Animals arrival | Week 0-2 | Acclimatization- Chow diet |
| (10 weeks old) | (2 weeks total) | |
| Start diet-induction | Week 2-6 | CDAA-HFD (A16092201; RD) |
| | (4 weeks total) | |
| Study period start | Week 6 | n = 10 animals; LEAN-CHOW |
| | | n = 48 animals; CDAA-HFD |
| Study Period | Week 14 | Animal sacrifice |
| termination | (Study period 8 | |
| | weeks total) | |

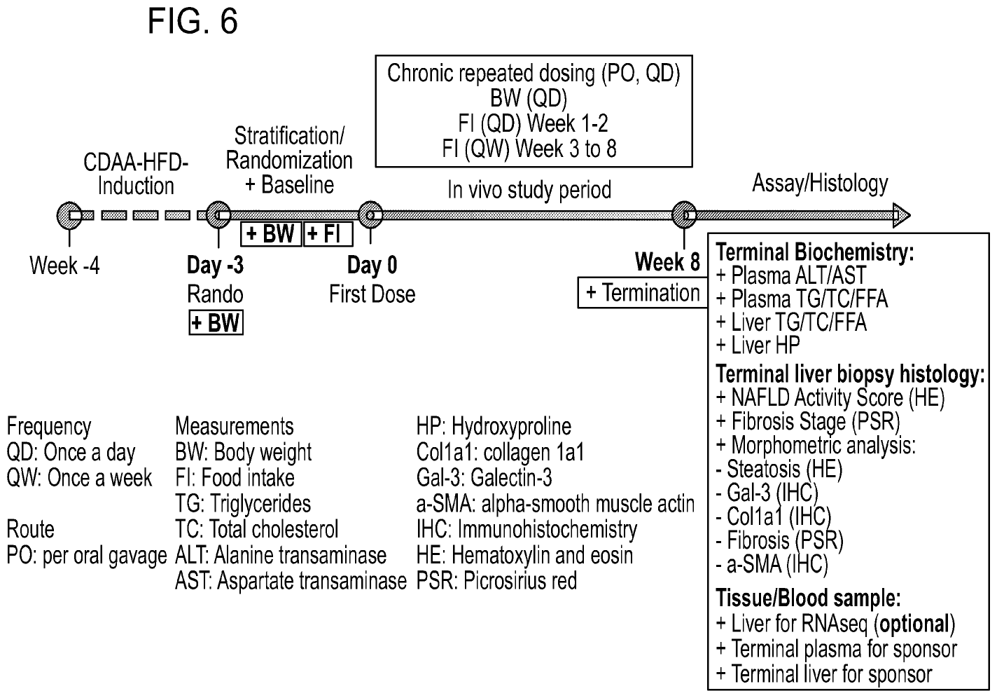

A representative diagram of the study outline is as shown in FIG. 6.

### Example 3. Effects of a Composition in DIAMOND Model of Liver Disease

### 1. Study Objective

To examine the effects of compositions described herein, for example, Composition A, a dosing solution will be tested in a Diet Induced Animal Model Of Non-alcoholic fatty liver Disease (DIAMOND) model of NAFLD, NASH, Fibrosis, and HCC. Composition A may include the following elements:

Composition A

| | |
|---|---|
| 20% | C16:1n7 |
| 70% | C18:1 |
| 5% | C16:0 |
| 5% | C18:2 |

### 2. Materials and Methods

### 2.1. Test Substances

Dosing solution may be prepared as described above or diluted as needed, for example with safflower oil, or mixed into Teklad 42% HFD chow.

### 2.2. Induction of Liver Disease

50 male DIAMOND™ mice will be raised to 8-12 weeks of age, and placed on either Western Diet (high sugar and high fat) to induce liver disease or Normal Chow control diet for 20 weeks.

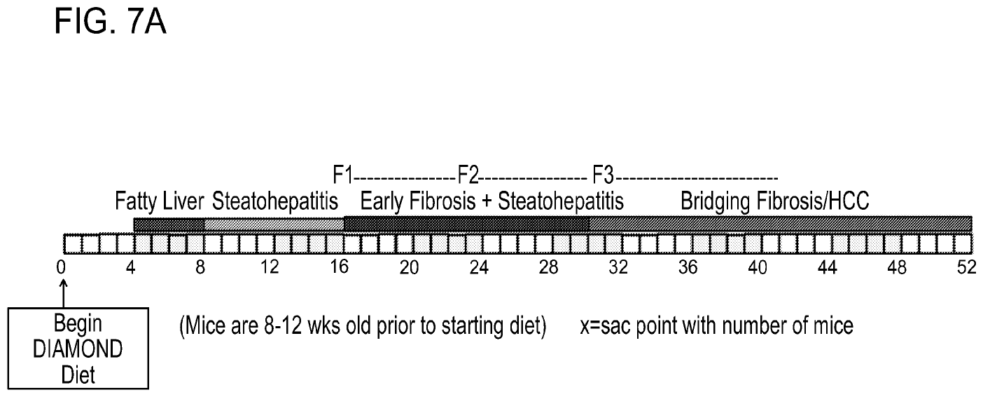

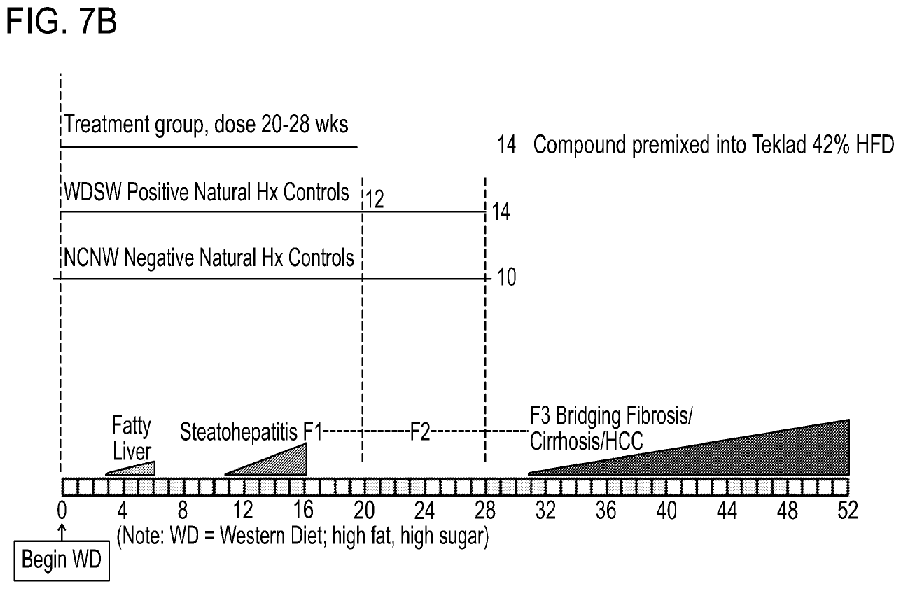

A representative diagram of the induction of liver disease is as shown in FIG. 7A.

### 2.3. Route of Administration

Test substance will be administered orally.

### 2.4. Dosing

Test substance will be administered once a day.

### 2.5. Measurement of Body Weight

Mice will be visually inspected daily. Body weight will be measured once weekly for the entire study period.

### 2.6. Biochemistry Measurement at Baseline

At baseline, positive controls will be sacrificed and tested for the following analytes: Fasting blood glucose and ketones, Lipid panel (total cholesterol (T chol), low-density lipoprotein (LDL), high-density lipoprotein (HDL), triglycerides (triggs)) and Hepatic Panel (albumin (ALB), alkaline phosphatase (ASP), aspartate aminotransferase (AST), alanine aminotransferase (ALT), blood urea nitrogen (BUN), gamma-glutamyl transferase (GGT) and total bilirubin (Tbil)).

### 2.7. Biochemistry Measurement at Study Conclusion.

At study conclusion, all treatment groups and positive and negative chow controls will be sacrificed and tested for the following analytes: Fasting blood glucose and ketones, Lipid panel (T chol, HDL, non-HDL, trigs) and Hepatic Panel (ALB, ALP, AST, ALT, BUN, GGT and Tbil).

### 2.8. Histological Analysis

A portion of the liver will be preserved in formalin for histology, and the rest will be snap-frozen and stored at −80 for future testing. Liver will be photographed and weighed at study conclusion.

Histology: for all animals in all groups, liver block preparation and slide cutting and staining. FFPE slides shall be cut and stained with H&E and Sirius Red for liver NAS scoring (fibrosis, steatosis grade and percentage, inflammation, ballooning, NAS, SAF activity score, Fibrosis NASH CRN Score, Perisinusoidal Fibrosis score, and NASH category), Dr. Pierre Bedossa will score, report and take representative photographs.

### 3. Experimental Design and Treatment

### 3.1. Study Groups

The animals will be randomized into 3 groups based on body weight.

### Group 1. Negative Natural Control

Group 1 (n=10) will be fed normal chow and administered no treatment.

### Group 2: Positive Natural Control

Group 2 (n=14) will be fed Western Diet (i.e., high fat, high sugar) and administered no treatment. At baseline 2 animals will be terminated for baseline biochemistry measurement.

### Group 3: Test Group

Group 3 (n=14) will be fed Western Diet and administered dosing solution premixed into Teklad 42% HFD chow.

### 3.2 Treatment Schedule

Test groups will be fed daily and study period will be 8 weeks long and conducted under IACUC protocol and carry out the study in AAALAC approved facilities.

A representative diagram of the study outline is as shown in FIG. 7B.

1. A method of treating or preventing a liver disease in a subject comprising administering a composition comprising a mixture of a first monounsaturated fatty acid and a second monounsaturated fatty acid, wherein the mixture has a melting point temperature of about −1.7° C. to about 10.7° C.

2. The method of claim 1, wherein the mixture has a melting point temperature of about 3° C. to about 9° C. or about 7° C. to about 9° C.

3. (canceled)

4. The method of claim 1,
(a) wherein the mixture comprises at least 80% by weight of the composition;
(b) wherein each of the first monounsaturated fatty acid and the second monounsaturated fatty has an iodine value of about 50 to about 130;
(c) wherein the first monounsaturated fatty acid has an acyl chain of 16 carbons or less; optionally, wherein the first monounsaturated fatty acid is selected from the group consisting of C12:1, C14:1 and C16:1, optionally, wherein the first monounsaturated fatty is C16:1n-7, C16:1n-6, C16:1n-5, C16:1n-4 or C16:1n-3;
(d) wherein the second monounsaturated fatty acid has an acyl chain of 18 carbons or more; optionally, wherein the second monounsaturated fatty acid is selected from the group consisting of C22:1, C20:1 and C18:1; optionally, wherein the second monounsaturated fatty acid is C18:1n-9;
(e) wherein the composition further comprises one or more additional monounsaturated fatty acids;
(f) wherein the double bond in the first monounsaturated fatty acid is a cis-isomer;
(g) wherein the double bond in the second monounsaturated fatty acid is a cis-isomer;
(h) wherein the first monounsaturated fatty acid is palmitoleic acid, and the second monounsaturated fatty acid is oleic acid;
(i) wherein the first monounsaturated fatty acid is present at up to about 40% by weight of the composition; about 18% to about 40% by weight of the composition; about 20% to about 40% by weight of the composition; about 18% to about 23% by weight of the composition; or about 20% to about 23% by weight of the composition;
(j) wherein the second monounsaturated fatty acid is present at up to about 80% by weight of the composition; about 40% to about 80% by weight of the composition; or about 60% to about 80% by weight of the composition;
(k) wherein the first monounsaturated fatty acid is present at about 20% to about 40% by weight of the composition and the second monounsaturated fatty acid is present at about 60% to about 80% by weight of the composition;
(l) wherein the first monounsaturated fatty acid is present at about 18% to about 23% by weight of the composition and the second monounsaturated fatty acid is present at about 60% to about 80% by weight of the composition; and/or
(m) wherein the first monounsaturated fatty acid is present at about 20% to about 23% by weight of the composition and the second monounsaturated fatty acid is present at about 60% to about 80% by weight of the composition; and/or
(n) wherein the composition is liquid at room temperature.

5-28. (canceled)

29. The method of claim 1,
(a) wherein the composition further comprises up to about 5%, about 10%, about 15%, about 20%, about 25% or about 30% by weight of saturated fatty acid and polyunsaturated fatty acid; or between about 5% and about 30%, or between about 10% and about 25% by weight of saturated fatty acid and polyunsaturated fatty acid;
(b) wherein the saturated fatty acid is selected from the group consisting of C16:0, C12:0, C14:0 and C10:0;
(c) wherein the saturated fatty acid is up to about 10%, about 15%, about 20% or about 25% by weight; or between about 2% and about 25% or between about 5% and about 20% by weight;
(d) wherein the polyunsaturated fatty acid is selected from the group consisting of C18:2, C18:3, C20:4, C20:5, C22:5 and C22:6;
(e) wherein the polyunsaturated fatty acid is up to about 10%, about 15% or about 20% by weight; or between about 2% and about 20% or between about 3% and about 15% by weight; and/or
(f) wherein the composition further comprises up to about 3% by weight of trace fatty acids.

30-33. (canceled)

34. The method of claim 29, wherein the composition comprises:
(a) about 10% to about 40% by weight of C16:1; about 40% to about 80% by weight of C18:1; up to about 15% of saturated fatty acids; and up to about 7% of polyunsaturated fatty acids;
(b) about 20% by weight of C16:1; about 60% by weight of C18:1; about 15% by weight of C16:0; and about 5% by weight of C18:2;
(c) about 10% to about 30% by weight of C16:1; about 60% to about 80% by weight of C18:1; up to about 7% of saturated fatty acids; and up to about 7% of polyunsaturated fatty acids; or
(d) about 20% by weight of C16:1; about 70% by weight of C18:1; about 5% by weight of C16:0; and about 5% by weight of C18:2.

35-39. (canceled)

40. The method of claim 1, wherein the liver disease is a fatty liver disease; liver inflammation, liver cell injury, hepatic fibrosis, cirrhosis, or liver cancer; optionally, wherein the liver disease is NAFLD, NASH or hepatocarcinoma.

41-44. (canceled)

45. The method of claim 1, wherein the composition is administered
(a) at a daily dose of about 0.01 to about 2 g, about 0.1 to about 1.5 g, or about 0.3 to about 1.3 g per kilogram of body weight;
(b) at a daily dose of 0.2 to 0.85 g per kilogram of body weight;
(c) at a daily dose of about 0.5 gram to about 60 grams;
(d) at a daily dose of about 2 grams, about 10 grams, about 20 grams, about 30 grams, about 40 grams, about 50 grams or about 60 grams;
(e) at a daily dose of about 1 gram, about 5 grams, about 10 grams, about 15 grams, about 20 grams, about 25 grams, about 30 grams, about 35 grams, about 40 grams, about 45 grams, about 50 grams or about 55 grams;
(f) for at least 8 weeks; and/or
(g) with one or more carrier fatty acids or wherein the composition further comprises one or more carrier fatty acids; optionally,
wherein the carrier fatty acids are selected from the group consisting of safflower oil, olive oil, palm oil, soybean oil, canola oil (rapeseed oil), corn oil, peanut oil, other vegetable oils and a combination thereof.

46-52. (canceled)

53. A composition comprising a mixture of a first monounsaturated fatty acid and a second monounsaturated fatty acid, wherein the mixture has a melting point temperature of about −1.7° C. to about 10.7° C.

54. The composition of claim 53, wherein the mixture has a melting point temperature of about 3° C. to about 9° C. or about 7° C. to about 9° C.

55. (canceled)

56. The composition of claim 53,
(a) wherein the mixture comprises at least 80% by weight of the composition;
(b) wherein each of the first monounsaturated fatty acid and the second monounsaturated fatty has an iodine value of about 50 to about 130;
(c) wherein the first monounsaturated fatty acid has an acyl chain of 16 carbons or less; optionally, wherein the first monounsaturated fatty acid is selected from the group consisting of C12:1, C14:1 and C16:1, optionally, wherein the first monounsaturated fatty is C16:1n-7, C16:1n-6, C16:1n-5, C16:1n-4 or C16:1n-3;
(d) wherein the second monounsaturated fatty acid has an acyl chain of 18 carbons or more; optionally, wherein the second monounsaturated fatty acid is selected from the group consisting of C22:1, C20:1 and C18:1; optionally, wherein the second monounsaturated fatty acid is C18:1n-9;
(e) wherein the composition further comprises one or more additional monounsaturated fatty acids;
(f) wherein the double bond in the first monounsaturated fatty acid is a cis-isomer;
(g) wherein the double bond in the second monounsaturated fatty acid is a cis-isomer;
(h) wherein the first monounsaturated fatty acid is palmitoleic acid, and the second monounsaturated fatty acid is oleic acid;
(i) wherein the first monounsaturated fatty acid is present at up to about 40% by weight of the composition; about 18% to about 40% by weight of the composition; about 20% to about 40% by weight of the composition; about 18% to about 23% by weight of the composition; or about 20% to about 23% by weight of the composition;
(j) wherein the second monounsaturated fatty acid is present at up to about 80% by weight of the composition; about 40% to about 80% by weight of the composition; or about 60% to about 80% by weight of the composition;
(k) wherein the first monounsaturated fatty acid is present at about 20% to about 40% by weight of the composition and the second monounsaturated fatty acid is present at about 60% to about 80% by weight of the composition;
(l) wherein the first monounsaturated fatty acid is present at about 18% to about 23% by weight of the composition and the second monounsaturated fatty acid is present at about 60% to about 80% by weight of the composition; and/or
(m) wherein the first monounsaturated fatty acid is present at about 20% to about 23% by weight of the composition and the second monounsaturated fatty acid is present at about 60% to about 80% by weight of the composition;
(n) wherein the composition is liquid at room temperature; and/or
(o) wherein the composition comprises one or more carrier fatty acids; optionally, wherein the carrier fatty acid is selected from the group consisting of safflower oil, olive oil, palm oil, soybean oil, canola oil (rapeseed oil), corn oil, peanut oil, other vegetable oils and a combination thereof.

57-80. (canceled)

81. The composition of claim 53,
(a) wherein the composition further comprises up to about 5%, about 10%, about 15%, about 20%, about 25% or about 30% by weight of saturated fatty acid and polyunsaturated fatty acid; or between about 5% and about 30%, or between about 10% and about 25% by weight of saturated fatty acid and polyunsaturated fatty acid;
(b) wherein the saturated fatty acid is selected from the group consisting of C16:0, C12:0, C14:0 and C10:0;
(c) wherein the saturated fatty acid is up to about 10%, about 15%, about 20% or about 25% by weight; or between about 2% and about 25% or between about 5% and about 20% by weight;
(d) wherein the polyunsaturated fatty acid is selected from the group consisting of C18:2, C18:3, C20:4, C20:5, C22:5 and C22:6;
(e) wherein the polyunsaturated fatty acid is up to about 10%, about 15% or about 20% by weight; or between about 2% and about 20% or between about 3% and about 15% by weight; and/or
(f) wherein the composition further comprises up to about 3% by weight of trace fatty acids.

82-85. (canceled)

86. The composition of claim 81, wherein the composition comprises:
(a) about 10% to about 40% by weight of C16:1; about 40% to about 80% by weight of C18:1; up to about 15% of saturated fatty acids; and up to about 7% of polyunsaturated fatty acids;
(b) about 20% by weight of C16:1; about 60% by weight of C18:1; about 15% by weight of C16:0; and about 5% by weight of C18:2;
(c) about 10% to about 30% by weight of C16:1; about 60% to about 80% by weight of C18:1; up to about 7% of saturated fatty acids; and up to about 7% of polyunsaturated fatty acids; or
(d) about 20% by weight of C16:1; about 70% by weight of C18:1; about 5% by weight of C16:0; and about 5% by weight of C18:2.

87-93. (canceled)

94. The composition of claim 53 for the treatment or prevention of liver disease wherein the composition comprises:
a) about 20% by weight of C16:1n-7;
b) about 70% by weight of C18:1n-9;
c) about 5% by weight of C16:0; and
d) about 5% by weight of C18:2.

95. The composition of claim 94, wherein the composition further comprises one or more carrier fatty acids selected from the group consisting of safflower oil, olive oil, palm oil, soybean oil, canola oil (rapeseed oil), corn oil, peanut oil, other vegetable oils and a combination thereof; optionally wherein the carrier fatty acid is safflower oil.

96. (canceled)
